(19) United States
(12) Patent Application Publication
(10) Pub. No.: US 2008/0070842 A1
(43) Pub. Date: Mar. 20, 2008

(54) RECOMBINANT BONE MORPHOGENETIC PROTEIN HETERODIMERS, COMPOSITIONS AND METHODS OF USE

(76) Inventors: David Israel, Concord, MA (US); Neil M. Wolfman, Dover, MA (US)

Correspondence Address: WYETH/FINNEGAN HENDERSON, LLP, 901 NEW YORK AVENUE, NW, WASHINGTON, DC, 20001-4413, US

(21) Appl. No.: 11/930,082

(22) Filed: Oct. 30, 2007

Related U.S. Application Data
(63) Continuation of application No. 10/375,150, filed on Feb. 28, 2003.
Application No. 10/375,150 is a division of application No. 09/780,601, filed on Feb. 9, 2001, now Pat. No. 6,593,109.
Application No. 09/780,601 is a division of application No. 08/469,411, filed on Jun. 6, 1995, now Pat. No. 6,190,880.
Application No. 08/469,411 is a continuation of application No. 07/989,847, filed on Nov. 27, 1992, now Pat. No. 5,866,364.
Application No. 07/989,847 is a continuation-in-part of application No. 07/864,692, filed on Apr. 7, 1992, now abandoned.
Application No. 07/864,692 is a continuation-in-part of application No. 07/787,496, filed on Nov. 4, 1991, now abandoned.

(51) Int. Cl. A61K 38/16; A61P 19/08; C07K 14/51

(52) U.S. Cl. 514/12; 530/350

(57) ABSTRACT

The present invention relates to methods for producing recombinant heterodimeric BMP proteins useful in the field of treating bone defects, healing bone injury and in wound healing in general. The invention also relates to the recombinant heterodimers and compositions containing them.

### CROSS REFERENCE TO RELATED APPLICATIONS

This application is a continuation-in-part of U.S. Ser. No. 07/864,692 filed Apr. 7, 1992, which is a continuation-in-part of U.S. Ser. No. 07/787,496 filed Nov. 4, 1991.

### FIELD OF THE INVENTION

The present invention relates to a series of novel recombinant heterodimeric proteins useful in the field of treating bone defects, healing bone injury and in wound healing in general. The invention also relates to methods for obtaining these heterodimers, methods for producing them by recombinant genetic engineering techniques, and compositions containing them.

### BACKGROUND OF THE INVENTION

In recent years, protein factors which are characterized by bone or cartilage growth inducing properties have been isolated and identified. See, e.g., U.S. Pat. No. 5,013,649, PCT published application WO90/11366; PCT published application WO91/05802 and the variety of references cited therein. See, also, PCT/US90/05903 which discloses a protein sequence termed OP-1, which is substantially similar to human BMP-7, and has been reported to have osteogenic activity.

A family of individual bone morphogenetic proteins (BMPs), termed BMP-2 through BMP-9 have been isolated and identified. Incorporated by reference for the purposes of providing disclosure of these proteins and methods of producing them are co-owned, co-pending U.S. patent application Ser. No. 721,847 and the related applications recited in its preamble. Of particular interest, are the proteins termed BMP-2 and BMP-4, disclosed in the above-referenced application; BMP-7, disclosed in SN 438,919; BMP-5, disclosed in Ser. No. 370,547 and Ser. No. 356,033; and BMP-6, disclosed in Ser. No. 370,544 and Ser. No. 347,559; and BMP-8, disclosed in Ser. No. 525,357. Additional members of the BMP family include BMP-1, disclosed in Ser. No. 655,578; BMP-9, disclosed in Ser. No. 720,590; and BMP-3, disclosed in Ser. No. 179,197 and PCT publication 89/01464. These applications are incorporated herein by reference for disclosure of these BMPs.

There remains a need in the art for other proteins and compositions useful in the fields of bone and wound healing.

### SUMMARY OF THE INVENTION

In one aspect, the invention provides a method for producing a recombinant heterodimeric protein having bone stimulating activity comprising culturing a selected host cell containing a polynucleotide sequence encoding a first selected BMP or fragment thereof and a polynucleotide sequence encoding a second selected BMP or fragment thereof. The resulting co-expressed, biologically active heterodimer is isolated from the culture medium.

According to one embodiment of this invention, the host cell may be co-transfected with one or more vectors containing coding sequences for one or more BMPs. Each BMP polynucleotide sequence may be present on the same vector or on individual vectors transfected into the cell. Alternatively, the BNPs or their fragments may be incorporated into a chromosome of the host cell. Additionally, a single transcription unit may encode single copy of two genes encoding a different BMP.

According to another embodiment of this invention, the selected host cell containing the two polypeptide encoding sequences is a hybrid cell line obtained by fusing two selected, stable host cells, each host cell transfected with, and capable of stably expressing, a polynucleotide sequence encoding a selected first or second BMP or fragment thereof.

In another aspect of the present invention, therefore, there are provided recombinant heterodimeric proteins comprising a protein or fragment of a first BMP in association with a protein or fragment of a second BMP. The heterodimer may be characterized by bone stimulating activity. The heterodimers may comprise a protein or fragment of BMP-2 associated with a protein or fragment of either BMP-5, BMP-6, BMP-7 or BMP-8; or a protein or fragment of BMP-4 associated with a protein or fragment of either BMP-5, BMP-6, BMP-7 or BMP-8. In further embodiments the heterodimers may comprise a protein or fragment of BMP-2 associated with a protein or fragment of either BMP-1, BMP-3 or BMP-4. BMP-4 may also form a heterodimer in association with BMP-1, BMP-2 or a fragment thereof. Still further embodiments may comprise heterodimers involving combinations of BMP-5, BMP-6, BMP-7 and BMP-8. For example, the heterodimers may comprise BMP-5 associated with BMP-6, BMP-7 or BMP-8; BMP-6 associated with BMP-7 or BMP-8; or BMP-7 associated with BMP-8. These heterodimers may be produced by co-expressing each protein in a selected host cell and isolating the heterodimer from the culture medium.

As a further aspect of this invention a cell line is provided which comprises a first polynucleotide sequence encoding a first BMP or fragment thereof and a second polynucleotide sequence encoding a second BMP or fragment thereof, the sequences being under control of one or more suitable expression regulatory systems capable of co-expressing the BMPs as a heterodimer. The cell line may be transfected with one or more than one polynucleotide molecule. Alternatively, the cell line may be a hybrid cell line created by cell fusion as described above.

Another aspect of the invention is a polynucleotide molecule or plasmid vector comprising a polynucleotide sequence encoding a first selected BMP or fragment thereof and a polynucleotide sequence encoding a second selected BMP or fragment thereof. The sequences are under the control of at least one suitable regulatory sequence capable of directing co-expression of each protein or fragment. The molecule may contain a single transcription unit containing a copy of both genes, or more than one transcription unit, each containing a copy of a single gene.

As still another aspect of this invention there is provided a method for producing a recombinant dimeric or heterodimeric protein having bone stimulating activity in a prokaryotic cell comprising culturing a selected host cell containing a polynucleotide sequence encoding a first selected BMP or fragment thereof; culturing a second selected host cell containing a polynucleotide sequence encoding a second selected BMP or fragment thereof; isolating monomeric forms of each BMP protein from the culture medium and co-assembling a monomer of the first protein with a monomer of the second protein. The first protein and the second protein may be the same or different BMPs. The resulting biologically active dimer or heterodimer is thereafter isolated from the mixture. Preferred cells are E. coli.

Thus, as further aspects of this invention recombinant BMP dimers or heterodimers produced in eukaryotic cells are provided, as well as suitable vectors or plasmids, and selected transformed cells useful in such a production method.

Other aspects and advantages of the present invention are described further in the following detailed description of preferred embodiments of the present invention.

### BRIEF DESCRIPTION OF THE FIGURES

FIG. 1 provides the DNA and amino acid sequences of human BMP-2 (SEQ ID NOs: 1 and 2).

FIG. 2 provides the DNA and amino acid sequences of human BMP-4 (SEQ ID NOs: 3 and 4).

FIG. 3 provides the DNA and amino acid sequences of human BMP-7 (SEQ ID NOs: 5 and 6).

FIG. 4 provides the DNA and amino acid sequences of human BMP-6 (SEQ ID NOs: 7 and 8).

FIG. 5 provides the DNA and amino acid sequences of human BMP-5 (SEQ ID NOs: 9 and 10).

FIG. 6 provides the DNA and amino acid sequences of human BMP-8 (SEQ ID NOs: 11 and 12).

FIG. 7 provides the DNA sequence of vector pALB2-781 containing the mature portion of the BMP-2 gene (SEQ ID NOs: 13 and 14).

FIG. 8 compares the activity of CHO BMP-2 and CHO BMP-2/7 in the W20 alkaline phosphatase assay.

FIG. 9 compares the activity of CHO BMP-2 and CHO BMP-2/7 in the BGP (osteocalcin) assay.

FIG. 10 provides a comparison of the W-20 activity of E. coli produced BMP-2 and BMP-2/7 heterodimer.

FIG. 11 depicts BMP-3 DNA and amino acid sequence.

FIG. 12 provides a comparison of BMP-2 and BMP-2/6 in the W-20 assay.

FIG. 13 provides a comparison of the in vivo activity of BMP-2/6 and BMP-2.

FIG. 14 provides a comparison of BMP-2, BMP-6 and BMP-2/6 in vivo activity.

### DETAILED DESCRIPTION OF THE INVENTION

The present invention provides a method for producing recombinant heterodimeric proteins having bone stimulating activity, as well as the recombinant heterodimers themselves, and compositions containing them for bone-stimulating or repairing therapeutic use.

As used throughout this document, the term ‘heterodimer’ is defined as a biologically-active protein construct comprising the association of two different BMP protein monomers or active fragments thereof joined through at least one covalent, disulfide linkage. A heterodimer of this invention may be characterized by the presence of between one to seven disulfide linkages between the two BMP component strands.

According to the present invention, therefore, a method for producing a recombinant BMP heterodimer according to this invention comprises culturing a selected host cell containing a polynucleotide sequence encoding a first selected BMP or a biologically active fragment thereof and a polynucleotide sequence encoding a second selected BMP or a fragment thereof. The resulting co-expressed, biologically active heterodimer is formed within the host cell, secreted therefrom and isolated from the culture medium. Preferred embodiments of methods for producing the heterodimeric proteins of this invention, are described in detail below and in the following examples. Preferred methods of the invention involve known recombinant genetic engineering techniques [See, e.g., Sambrook et al, “Molecular Cloning. A Laboratory Manual.”, 2d edition, Cold Spring Harbor Laboratory, Cold Spring Harbor, N.Y. (1989)]. However, other methods, such as conventional chemical synthesis may also be useful in preparing a heterodimer of this invention.

BMP heterodimers generated by this method are produced in a mixture of homodimers and heterodimers. This mixture of heterodimers and homodimers may be separated from contaminants in the culture medium by resort to essentially conventional methods, such as classical protein biochemistry or affinity antibody columns specific for one of the BMPs making up the heterodimer. Additionally, if desired, the heterodimers may be separated from homodimers in the mixture. Such separation techniques allow unambiguous determination of the activity of the heterodimeric species. Example 4 provides one presently employed purification scheme for this purpose.

Preferably the recombinant heterodimers of this invention produced by these methods involve the BMPs designated human BMP-2, human BMP-4, human BMP-5, human BMP-6, human BMP-7 and BMP-8. However, BMP-3 has also been determined to form an active heterodimer with BMP-2. other species of these BMPs as well as BMPs than those specifically identified above may also be employed in heterodimers useful for veterinary, diagnostic or research use. However, the human proteins, specifically those proteins identified below, are preferred for human pharmaceutical uses.

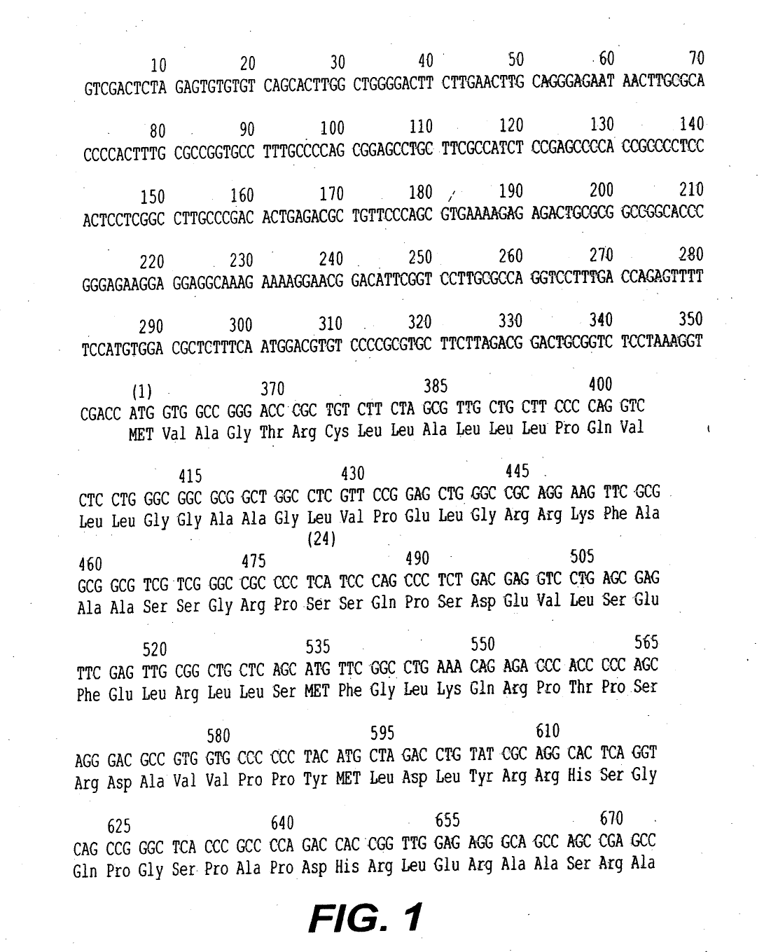

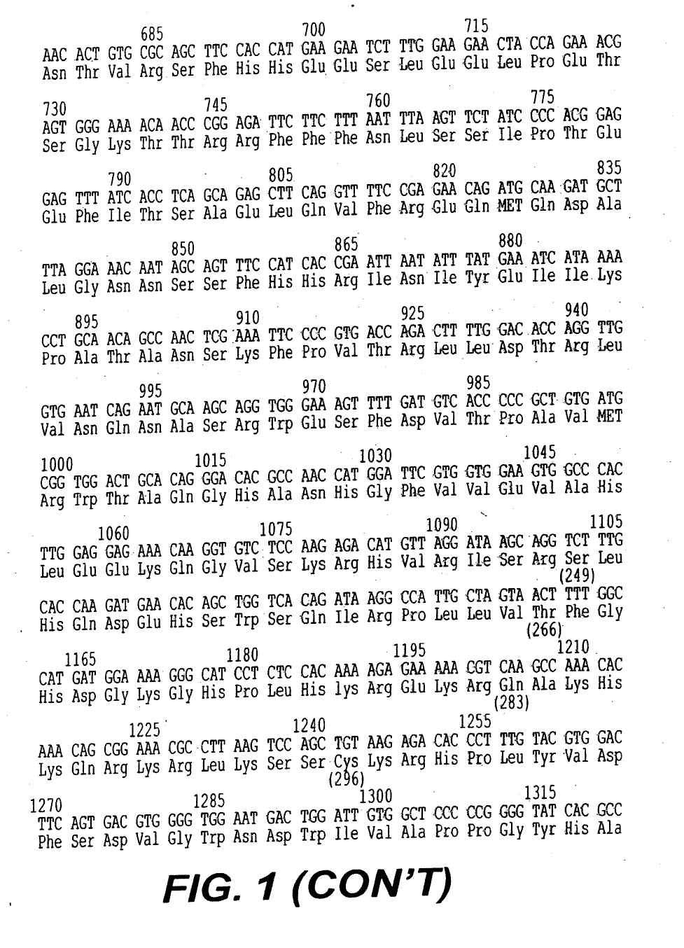

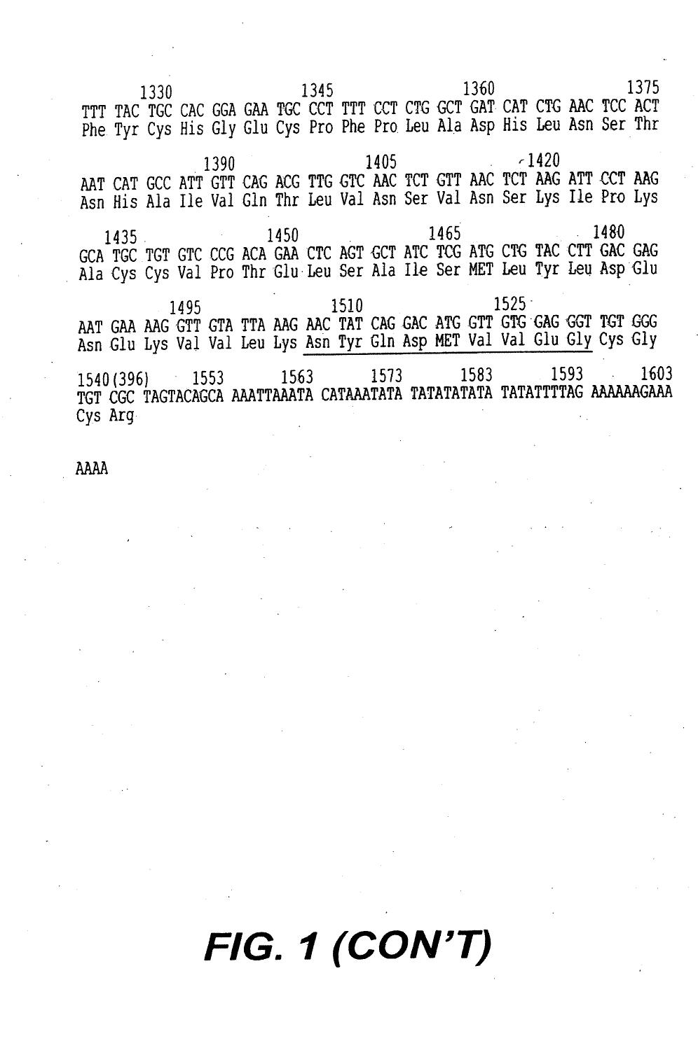

Human BMP-2 is characterized by containing substantially the entire sequence, or fragments, of the amino acid sequence and DNA sequence disclosed in FIG. 1. Human BMP-2 proteins are further characterized as disulfide-linked dimers and homodimers of mature BMP-2 subunits. Recombinantly-expressed BMP-2 subunits include protein species having heterogeneous amino termini. One BMP-2 subunit is characterized by comprising amino acid #249 (Ser)-#396 (Arg) of FIG. 1 (SEQ ID NOs: 1 and 2). Another BMP-2 subunit is characterized by comprising amino acid #266 (Thr)-#396-(Arg) of FIG. 1. Another BMP-2 subunit is characterized by comprising amino acid #296 (Cys)-#396 (Arg) of FIG. 1. A mature BMP-2 subunit is characterized by comprising amino acid #283 (Gln)-#396 (Arg) of FIG. 1. This latter subunit is the presently most abundant protein species which results from recombinant expression of BMP-2 (FIG. 1). However, the proportions of certain species of BMP-2 produced may be altered by manipulating the culture conditions. BMP-2 may also include modifications of the sequences of FIG. 1, e.g., deletion of amino acids #241-280 and changing amino acid #245 Arg to Ile, among other changes.

As described in detail in United States Patent Application Ser. No. 721,847, incorporated by reference herein, human BMP-2 may be produced by culturing a cell transformed with a DNA sequence comprising the nucleotide coding sequence from nucleotide #356 to #1543 in FIG. 1 and recovering and purifying from the culture medium one or more of the above-identified protein species, substantially free from other proteinaceous materials with which it is co-produced. Human BMP-2 proteins are characterized by the ability to induce bone formation. Human BMP-2 also has in vitro activity in the W20 bioassay. Human BMP-2 is further characterized by the ability to induce cartilage formation. Human BMP-2 may be further characterized by the ability to demonstrate cartilage and/or bone formation activity in the rat bone formation assay described in the above-referenced application.

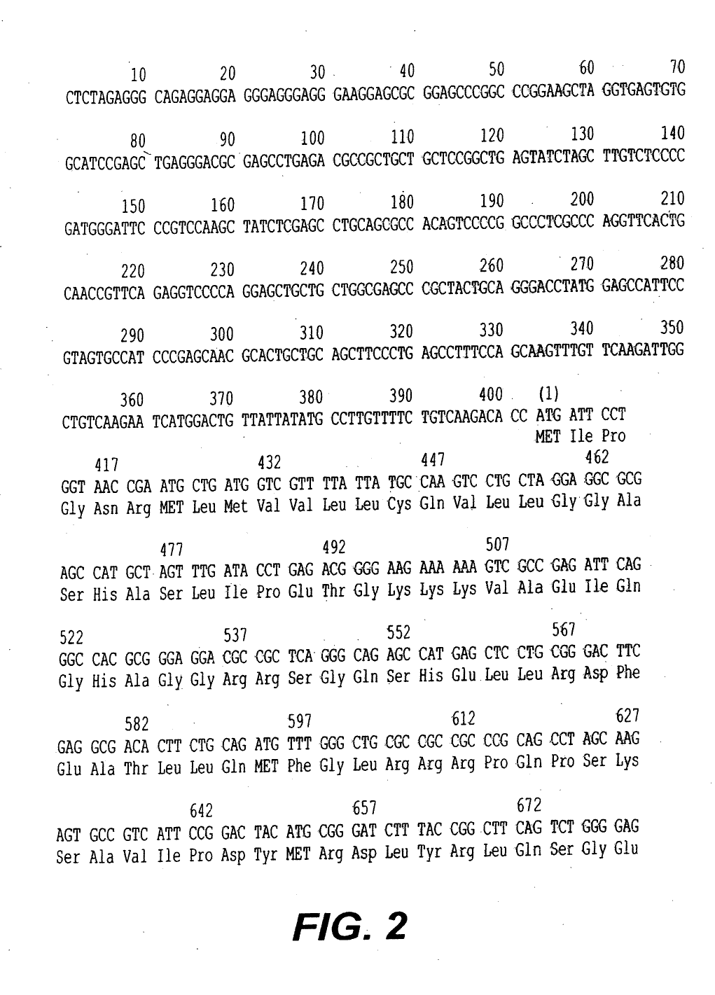

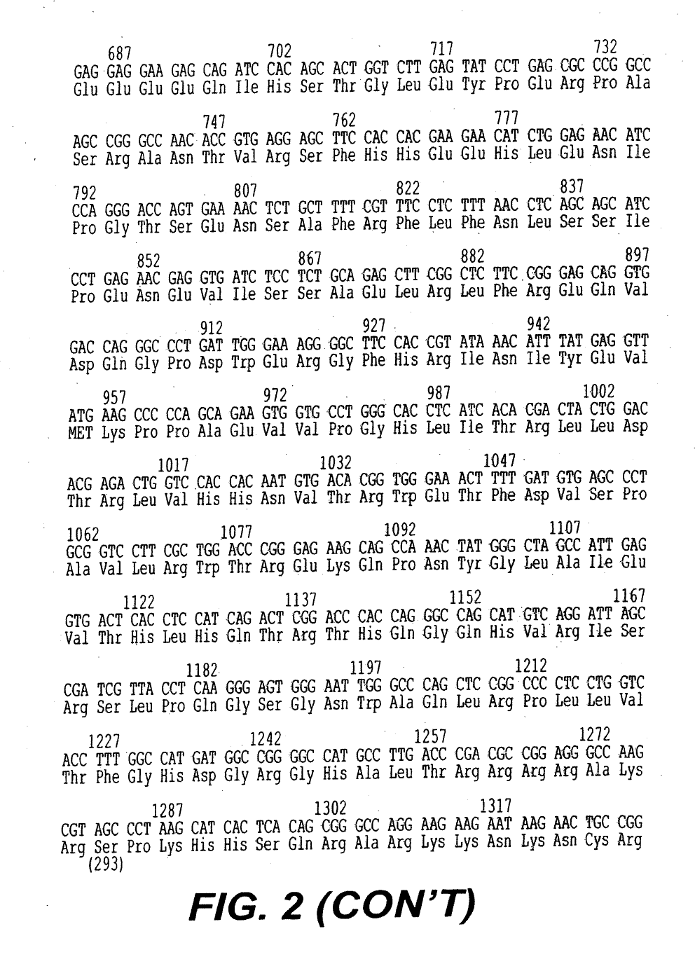

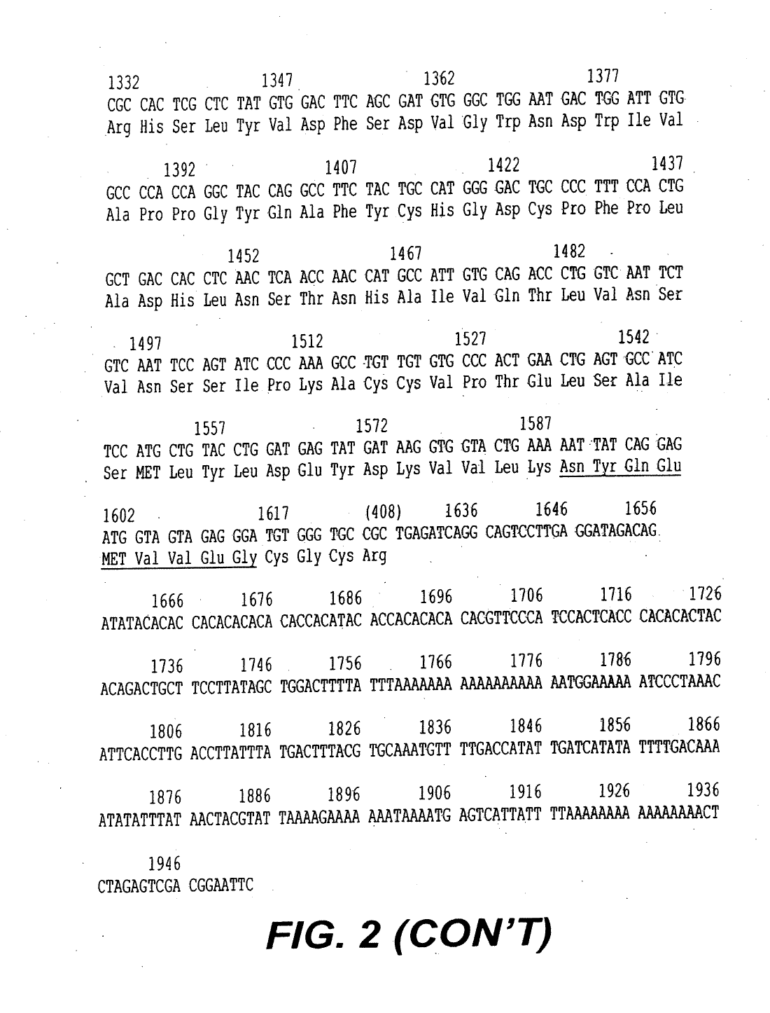

Human BMP-4 is characterized by containing substantially the entire sequence, or fragments, of the amino acid sequence and DNA sequence disclosed in FIG. 2 (SEQ ID NOs: 3 and 4). Human BMP-4 proteins are further characterized as disulfide-linked diners and homodimers of mature BMP-4 subunits. Recombinantly-expressed BMP-4 subunits may include protein species having heterogeneous amino termini. A mature subunit of human BMP-4 is characterized by an amino acid sequence comprising amino acids #293 (Ser)-#408 (Arg) of FIG. 2. Other amino termini of BMP-4 may be selected from the sequence of FIG. 2. Modified versions of BMP-4, including proteins further truncated at the amino or carboxy termini, may also be constructed by resort to conventional mutagenic techniques.

As disclosed in above-incorporated patent application Ser. No. 721,847, BMP-4 may be produced by culturing a cell transformed with a DNA sequence comprising the nucleotide coding sequence from nucleotide #403 to nucleotide #1626 in FIG. 2 and recovering and purifying from the culture medium a protein containing the amino acid sequence from amino acid #293 to #408 as shown in FIG. 2, substantially free from other proteinaceous materials with which it is co-produced. BMP-4 proteins are capable of inducing the formation of bone. BMP-4 proteins are capable of inducing formation of cartilage. BMP-4 proteins are further characterized by the ability to demonstrate cartilage and/or bone formation activity in the rat bone formation assay.

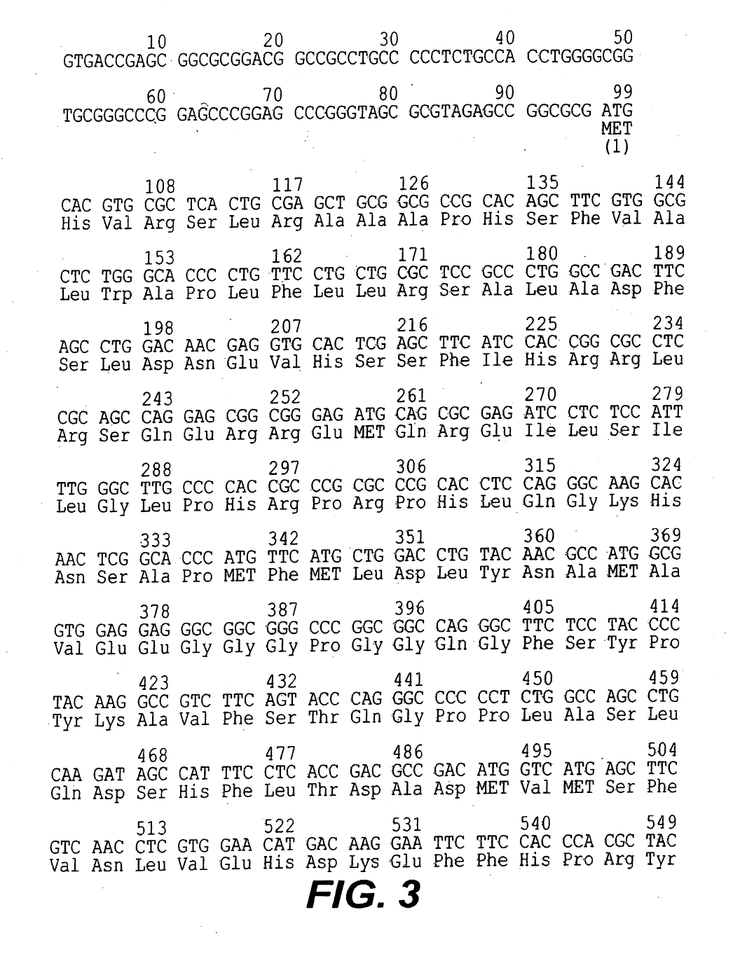

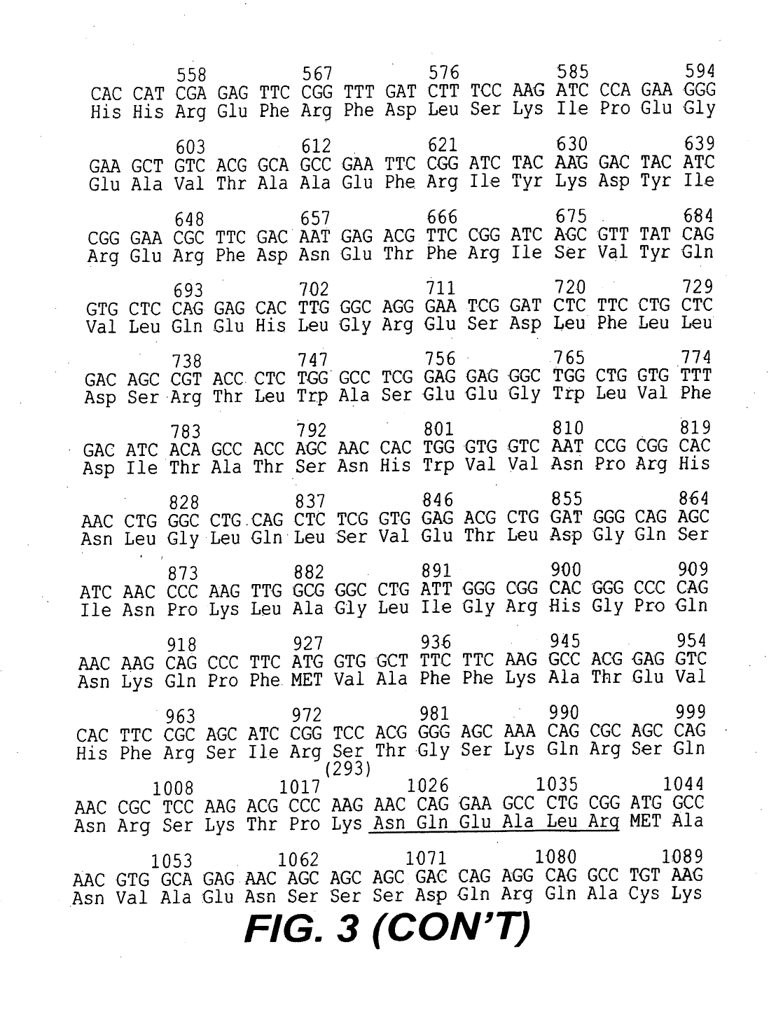

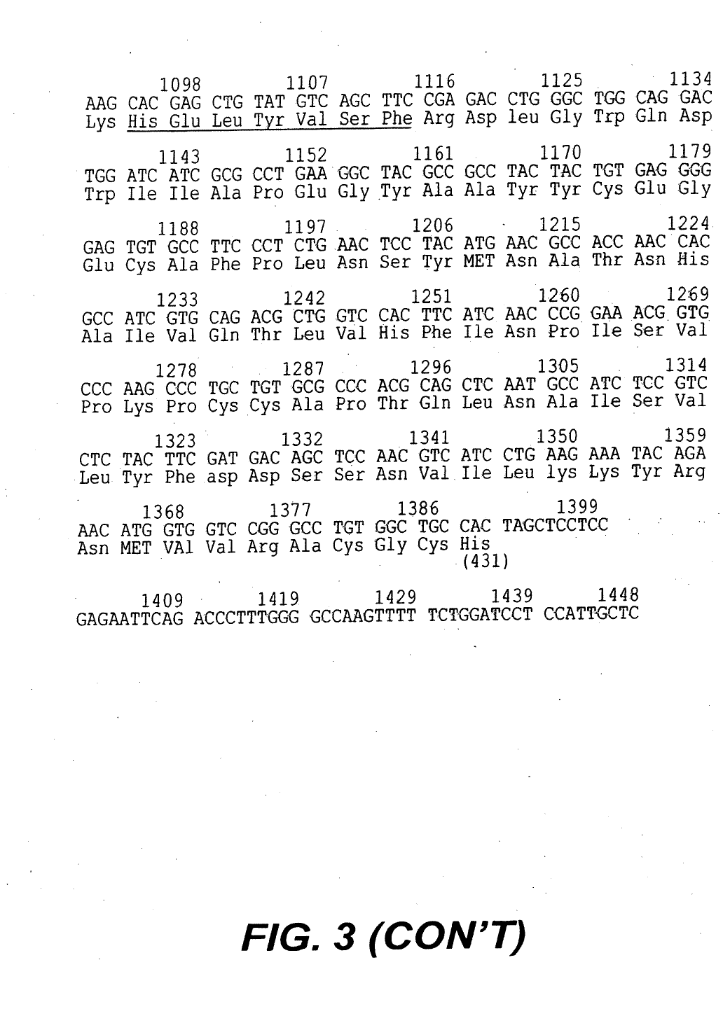

Human BMP-7 is characterized by containing substantially the entire sequence, or fragments, of the amino acid sequence and DNA sequence disclosed in FIG. 3. Human BMP-7 proteins are further characterized as disulfide-linked diners and homodimers of mature BMP-7 subunits. Recombinantly-expressed BMP-7 subunits include protein species having heterogeneous amino termini. One BMP-7 subunit is characterized by comprising amino acid #293 (Ser)-#431 (His) of FIG. 3 (SEQ ID NOs: 5 and 6). This subunit is the most abundantly formed protein produced by recombinant expression of the BMP-7 sequence. Another BMP-7 subunit is characterized by comprising amino acids #300 (Ser)-#431 (His) of FIG. 3. Still another BMP-7 subunit is characterized by comprising amino acids #316 (Ala)-#431 (His) of FIG. 3. Other amino termini of BMP-7 may be selected from the sequence of FIG. 3. Similarly, modified versions, including proteins further truncated at the amino or carboxy termini, of BMP-7 may also be constructed by resort to conventional mutagenic techniques.

As disclosed in above-incorporated patent application Ser. No. 438,919, BMP-7 may be produced by culturing a cell transformed with a DNA sequence comprising the nucleotide coding sequence from nucleotide #97 to nucleotide #1389 in FIG. 3 and recovering and purifying from the culture medium a protein containing the amino acid sequence from amino acid #293 to #431 as shown in FIG. 3, substantially free from other proteinaceous or contaminating materials with which it is co-produced. These proteins are capable of stimulating, promoting, or otherwise inducing cartilage and/or bone formation.

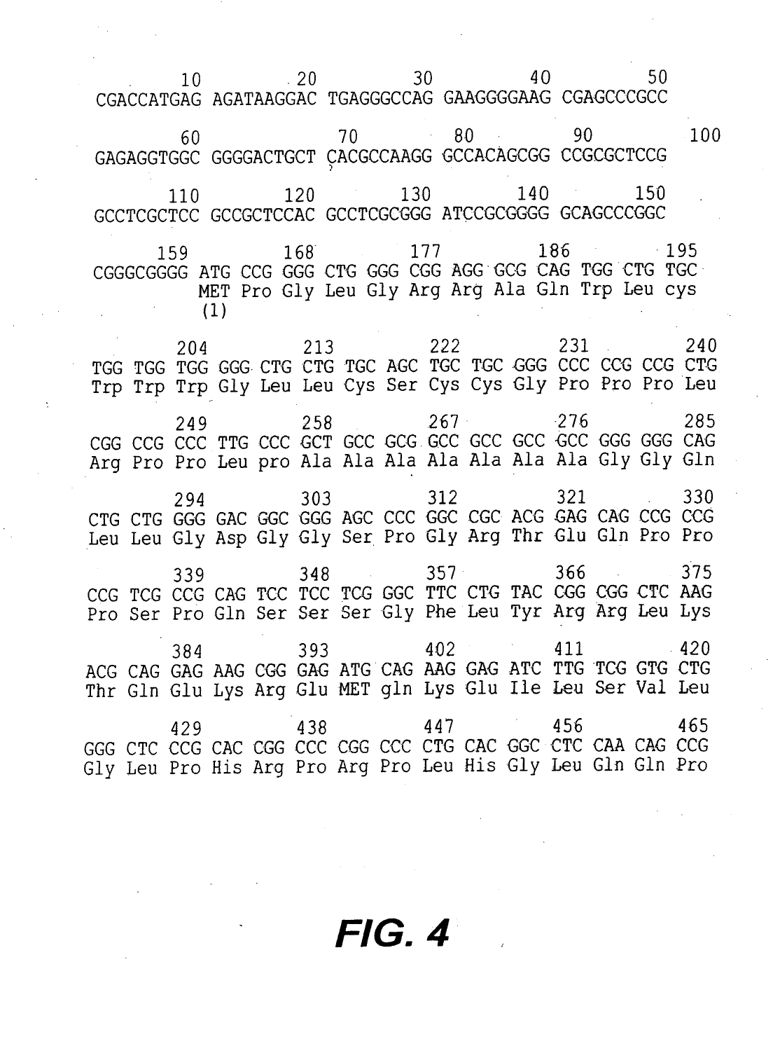

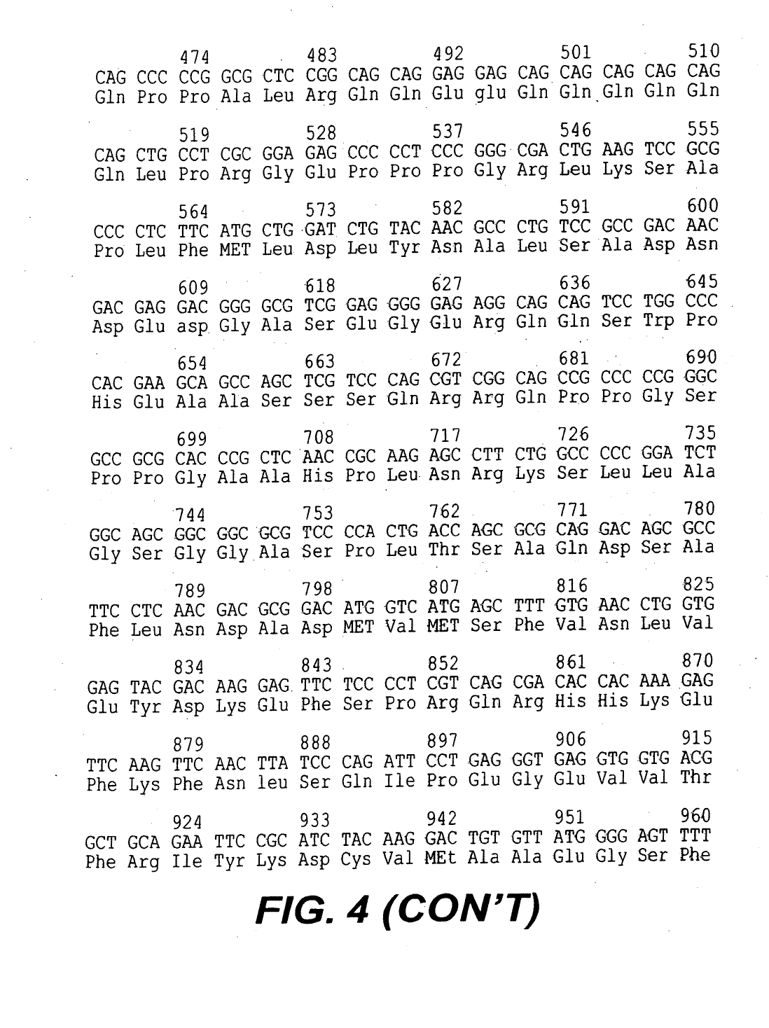

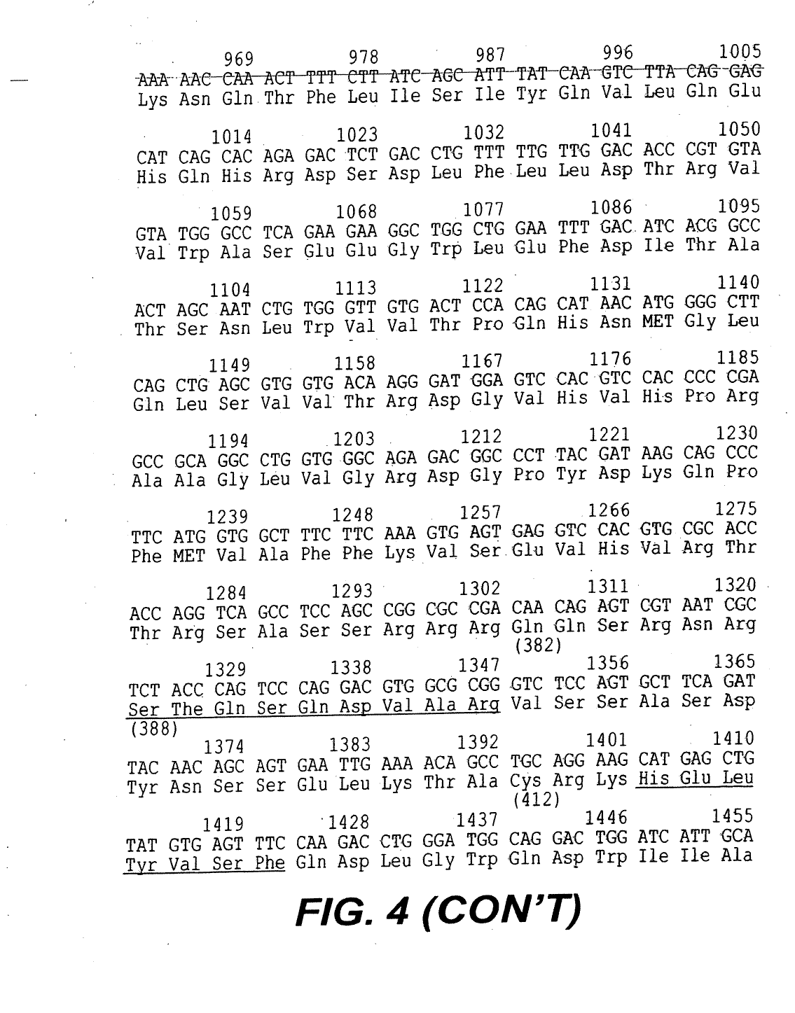

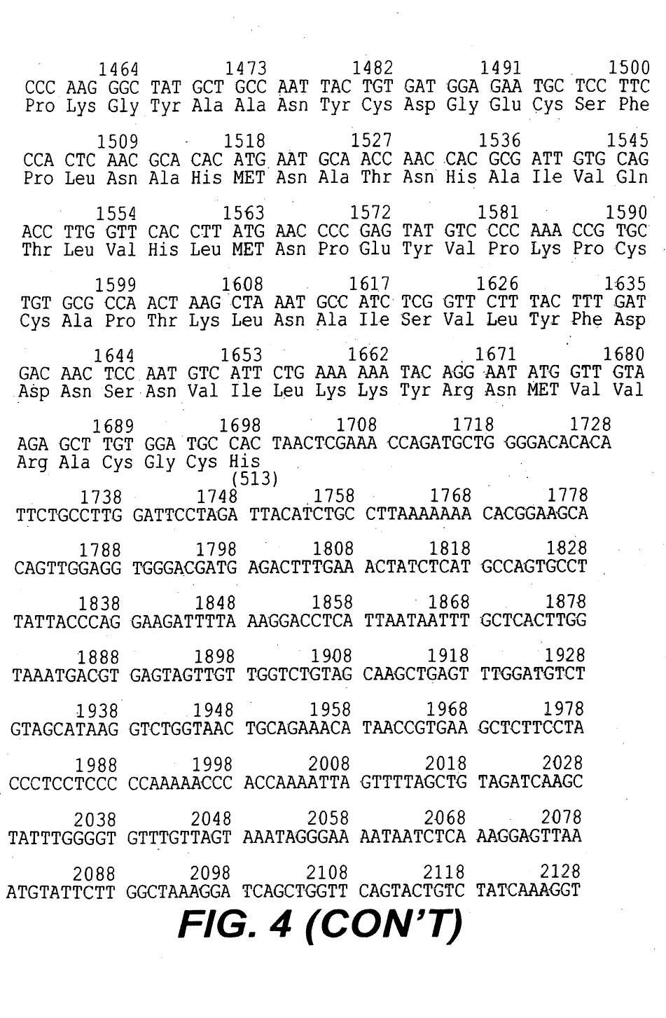

Human BMP-6 is characterized by containing substantially the entire sequence, or fragments, of the amino acid sequence and DNA sequence disclosed in FIG. 4. Human BMP-6 proteins are further characterized as disulfide-linked dimers of mature BMP-6 subunits. Recombinantly-expressed BMP-6 subunits may include protein species having heterogeneous amino termini. One BMP-6 subunit is characterized by comprising amino acid #375 (Ser)-#513 (His) of FIG. 4 (SEQ ID NOs: 7 and 8). Other amino termini of BMP-6 may be selected from the sequence of FIG. 4. Modified versions, including proteins further truncated at the amino or carboxy termini, of BMP-6 may also be constructed by resort to conventional mutagenic techniques.

As described in detail in United States Patent Application Ser. No. 490,033, incorporated by reference herein, human BMP-6 may be produced by culturing a cell transformed with a DNA sequence comprising the nucleotide coding sequence from nucleotide #160 to #1698 in FIG. 4 and recovering and purifying from the culture medium a protein comprising amino acid #375 to #513 of FIG. 4, substantially free from other proteinaceous materials or other contaminating materials with which it is co-produced. Human BMP-6 may be further characterized by the ability to demonstrate cartilage and/or bone formation activity in the rat bone formation assay.

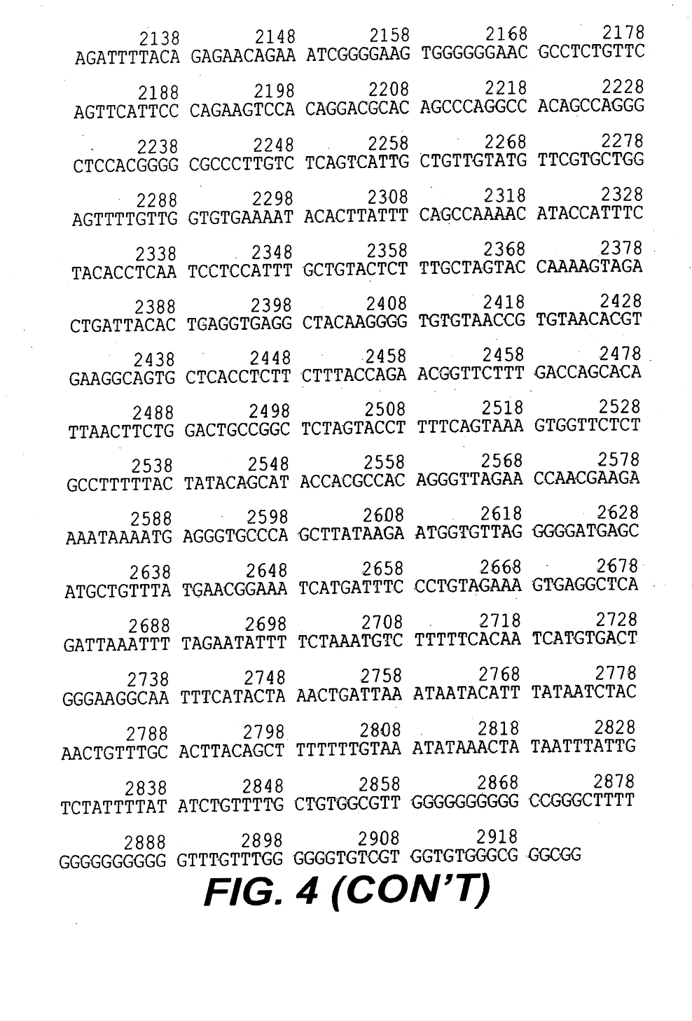

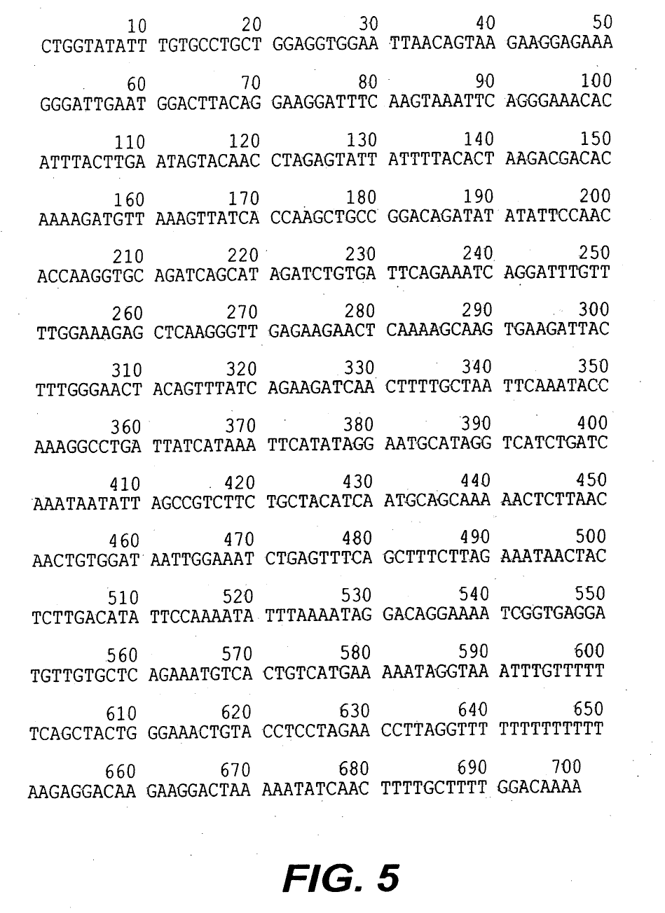

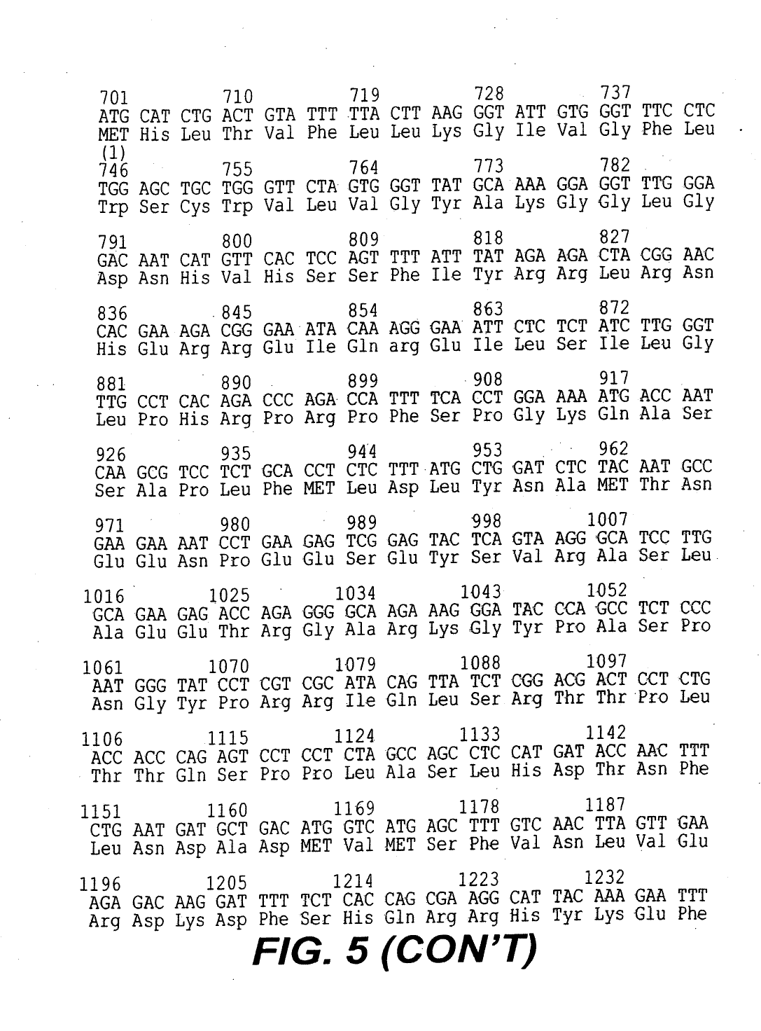

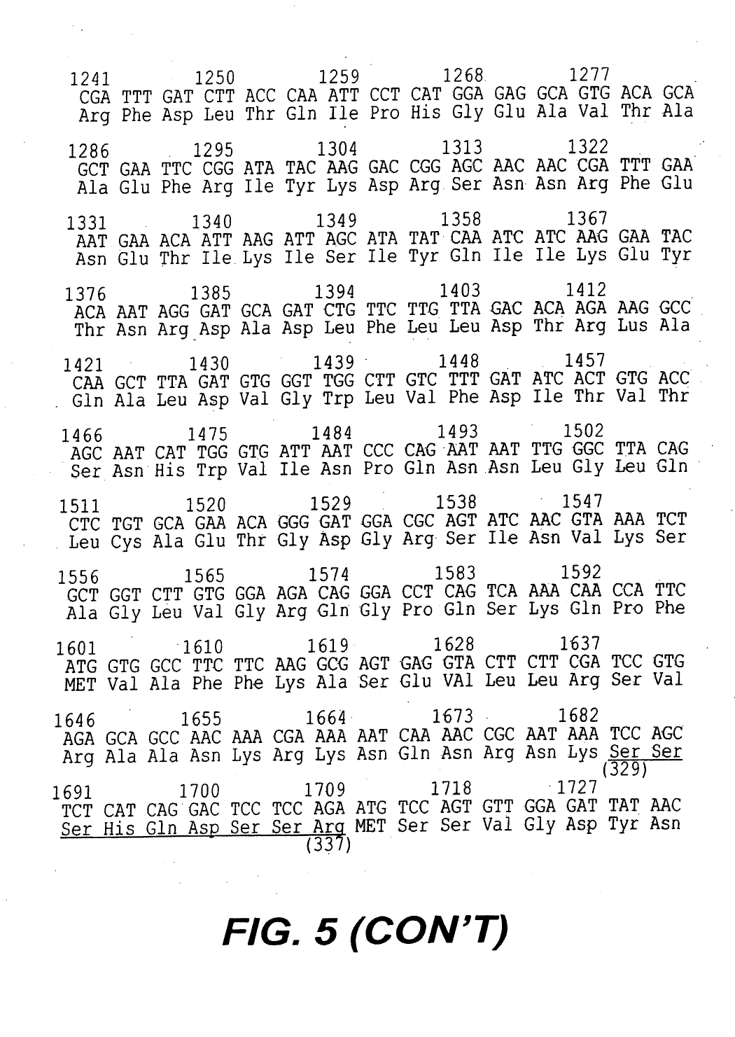

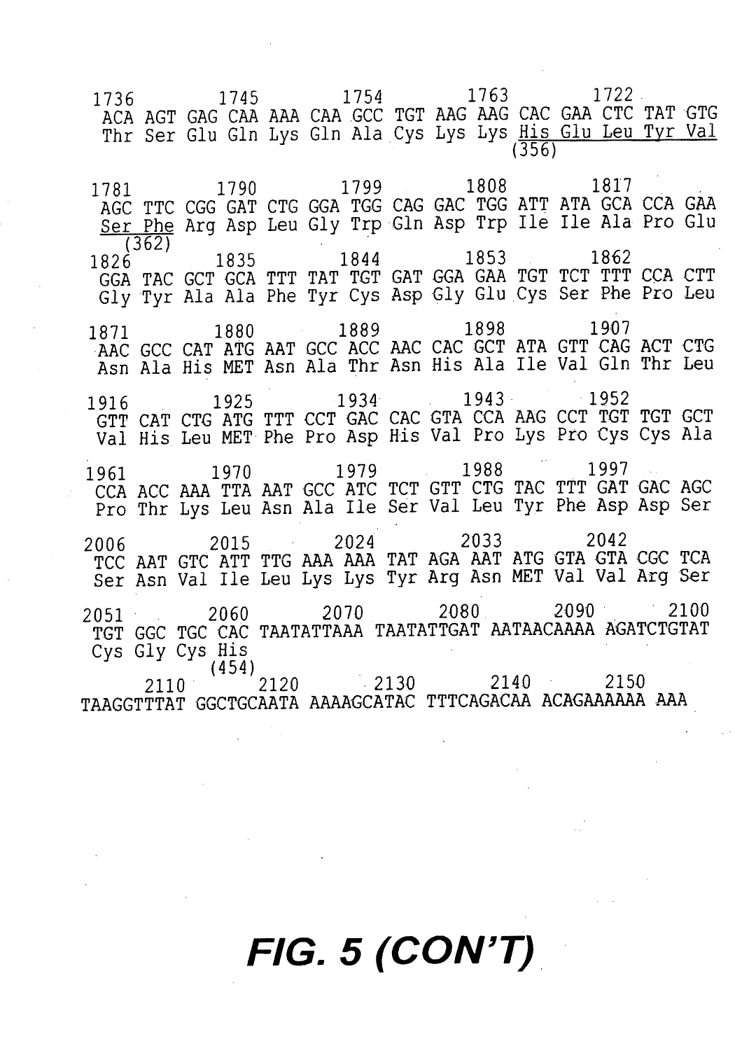

Human BMP-5 is characterized by containing substantially the entire sequence, or fragments, of the amino acid sequence and DNA sequence disclosed in FIG. 5 (SEQ ID NOs: 9 and 10). Human BMP-5 proteins are further characterized as disulfide-linked dimers of mature BMP-5 subunits. Recombinantly-expressed BMP-5. subunits may include protein species having heterogeneous amino termini. One BMP-5 subunit is characterized by comprising amino acid #329 (Ser)-#454 (His) of FIG. 5. Other amino termini of BMP-5 may be selected from the sequence of FIG. 5. Modified versions, including proteins further truncated at the amino or carboxy termini, of BMP-5 may also be constructed by resort to conventional mutagenic techniques.

As described in detail in U.S. patent application Ser. No. 588,227, incorporated by reference herein, human BMP-5 may be produced by culturing a cell transformed with a DNA sequence comprising the nucleotide coding sequence from nucleotide #701 to #2060 in FIG. 5 and recovering and purifying from the culture medium a protein comprising amino acid #329 to #454 of FIG. 5, substantially free from other proteinaceous materials or other contaminating materials with which it is co-produced. Human BMP-5 may be further characterized by the ability to demonstrate cartilage and/or bone formation activity in the rat bone formation assay described in the above-referenced application.

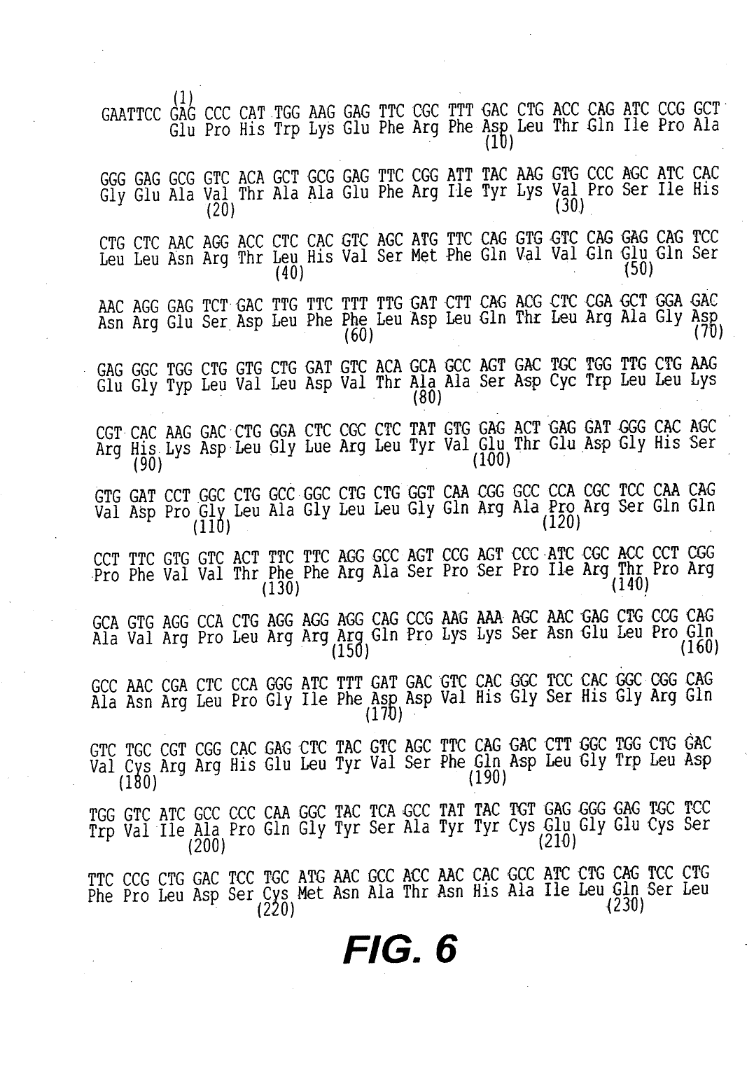

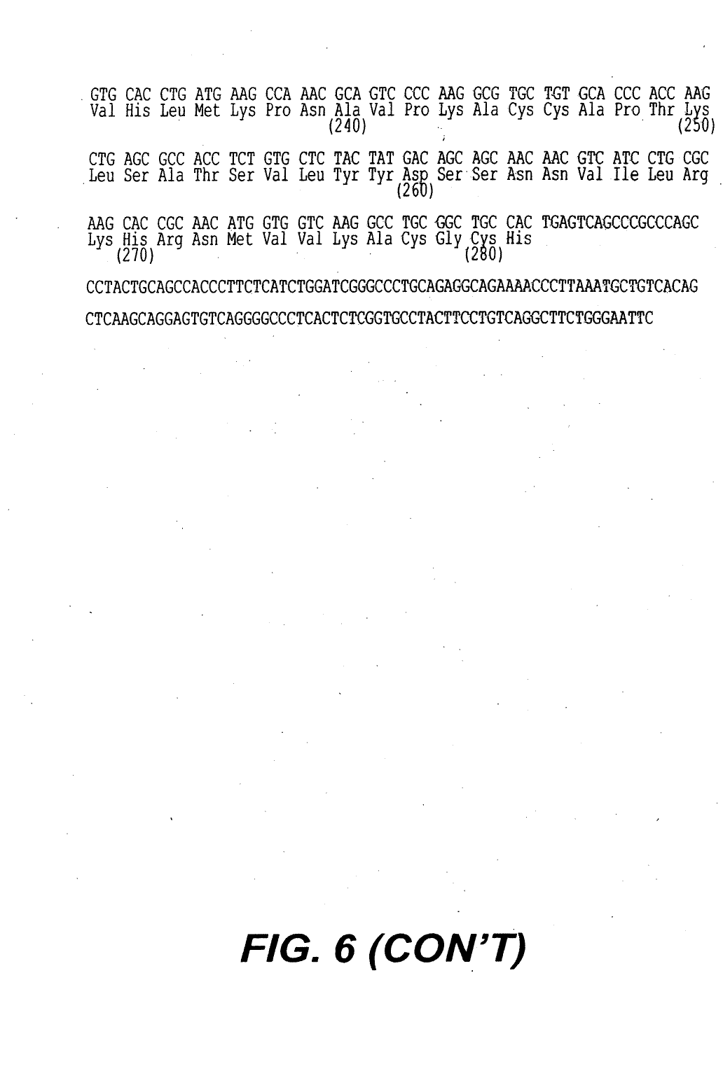

Human BMP-8 is characterized by containing substantially the entire sequence, or fragments, of the amino acid sequence and DNA sequence disclosed in FIG. 6. Human BMP-8 proteins may be further characterized as disulfide-linked dimers of mature BMP-8 subunits. Recombinantly-expressed BMP-8 subunits may include protein species having heterogeneous amino termini. A BMP-8 sequence or subunit sequence comprises amino acid #143 (Ala)-#281 (His) of FIG. 6 (SEQ ID NOs: 11 and 12). Other amino termini of BMP-8 may be selected from the sequence of FIG. 6. Modified versions, including proteins further truncated at the amino or carboxy termini, of BMP-8 may also be constructed by resort to conventional mutagenic techniques.

As described generally in U.S. patent application Ser. No. 525,357, incorporated by reference herein, and as further described herein, human BMP-8 may be produced by culturing a cell transformed with a DNA sequence comprising the nucleotide coding sequence from nucleotide #1 to #850 in FIG. 6 and recovering and purifying from the culture medium a protein comprising amino acid #143 to #281 of FIG. 6, or similar amino acid sequences with heterogenous N-termini, substantially free from other proteinaceous materials or other contaminating materials with which it is co-produced. This BMP-8 may also be produced in E. coli by inserting into a vector the sequence encoding amino acid #143 to #281 of FIG. 6 with a Met inserted before amino acid #143. Human BMP-8 may be further characterized by the ability to demonstrate cartilage and/or bone formation activity in the rat bone formation assay.

Each above described BMP protein in its native, non-reduced dimeric form may be further characterized by an apparent molecular weight on a 12% Laemmli gel ranging between approximately 28 kD to approximately 40 kb. Analogs or modified versions of the DNA and amino acid sequences described herein which provide proteins or active fragments displaying bone stimulating or repairing activity in the rat bone formation assay described below in Example 9, are also classified as suitable BMPs for use in this invention, further provided that the proteins or fragments contain one or more Cys residues for participation in disulfide linkages. Useful modifications of these sequences may be made by one of skill in the art with resort to known recombinant genetic engineering techniques. Production of these BMP sequences in mammalian cells produces homodimers, generally mixtures of homodimers having heterologous N termini. Production of these BMP sequences in E. coli produces monomeric protein species.

Thus, according to this invention one recombinant heterodimer of the present invention comprises the association of a human BMP-2, including, e.g., a monomeric strand from a mature BMP-2 subunit as described above or an active fragment thereof, bound through one or up to seven covalent, disulfide linkages to a human BMP-5 including, e.g., a monomeric strand from a mature BMP-5 subunit as described above or an active fragment thereof. Another recombinant heterodimer of the present invention comprises the association of a human BMP-2, as described above., bound through one or up to seven covalent, disulfide linkages to a human BMP-6, including, e.g., a monomeric strand from a BMP-6 subunit as described above or an active fragment thereof. Another recombinant heterodimer of the present invention comprises the association of a human BMP-2, as described above, bound through one or up to seven covalent, disulfide linkages to a human BMP-7, including, e.g., a monomeric strand of a BMP-7 subunit as described above or an active fragment thereof. Another recombinant heterodimer of the present invention comprises the association of a human BMP-2, as described above, bound through one or up to seven covalent, disulfide linkages to a human BMP-8, including, e.g., a monomeric strand of a BMP-8 subunit as described above or an active fragment thereof.

Still another recombinant heterodimer of the present invention comprises the association of a human BMP-4, including, e.g., a monomeric strand of a BMP-4 subunit as described above or an active fragment thereof, bound through one or up to seven covalent, disulfide linkages to a human BMP-5, as described above. Another recombinant heterodimer of the present invention comprises the association of a human BMP-4, as described above, bound through one or more covalent, disulfide linkages to a human BMP-6, as described above. Another recombinant heterodimer of the present invention comprises the association of a human BMP-4, as described above bound through one or more covalent, disulfide linkages to a human BMP-7, as described above. Another recombinant heterodimer of the present invention comprises the association of a human BMP-4, as described above, bound through one or more covalent, disulfide linkages to a human BMP-8, as described above.

A further recombinant heterodimer of the present invention comprises the association of a human BMP-2, including, e.g., a monomeric strand from a mature BMP-2 subunit as described above or an active fragment thereof, bound through at least one disulfide linkage to a human BMP-3 including, e.g., a monomeric strand from a mature BMP-3 subunit as described above or an active fragment thereof. Another recombinant heterodimer of the present invention comprises the association of a human BMP-2, as described above, bound through at least one disulfide linkage to a human BMP-4, including, e.g., a monomeric strand from a BMP-4 subunit as described above or an active fragment thereof. Another recombinant heterodimer of the present invention comprises the association of a human BMP-5, as described above, bound through at least one disulfide linkage to a human BMP-6, including, e.g., a monomeric strand of a BMP-6 subunit as described above or an active fragment thereof. Another recombinant heterodimer of the present invention comprises the association of a human BMP-5, as described above, bound through at least one disulfide linkage to a human BMP-7, including, e.g., a monomeric strand of a BMP-7 subunit as described above or an active fragment thereof. In addition, human BMP-5 may be associated with human BMP-8 bound through at least one disulfide linkage to a human BMP-8 subunit or active fragment thereof.

Still another recombinant heterodimer of the present invention comprises the association of a human BMP-6, including, e.g., a monomeric strand of a BMP-6 subunit as described above or an active fragment thereof, bound through at least one disulfide linkage to a human BMP-7, as described above. Another recombinant heterodimer of the present invention comprises the association of a human BMP-6, as described above, bound through one or more covalent, disulfide linkages to a human BMP-8, as described above. Another recombinant heterodimer of the present invention comprises the association of a human BMP-7, as described above bound through one or more covalent, disulfide linkages to a human BMP-8, as described above.

The disulfide linkages formed between the monomeric strands of the BMPs may occur between one Cys on each strand. Disulfide linkages may form between two Cys on each BMP. Disulfide linkages may form between three Cys on each BMP. Disulfide linkages may form between four Cys on each BMP. Disulfide linkages may form between five Cys on each BMP. Disulfide linkages may form between six Cys on each BMP. Disulfide linkages may form between seven Cys on each BMP. These disulfide linkages may form between adjacent Cys on each BMP or between only selected Cys interspersed within the respective protein sequence. Various heterodimers having the same BMP component strands may form with different numbers of disulfide linkages. Various heterodimers having the same BMP component strands may form with disulfide bonds at different Cys locations. Different heterodimers encompassed by this invention having the same BMP components may differ based upon their recombinant production in mammalian cells, bacterial cells, insect or yeast cells.

These recombinant heterodimers may be characterized by increased alkaline phosphatase activity in the W20 mouse stromal cell line bioassay (Example 8) compared to the individual BMP homodimers, one strand of which forms each heterodimer. Further, these heterodimers are characterized by greater activity in the W20 bioassay than is provided by simple mixtures of the individual BMP diners. Preliminary characterization of heterodimers measured on the W20 bioassay have demonstrated that heterodimers of BMP-2 with BNP-5, BMP-6 or BMP-7 are very active. Similarly, heterodimers of BMP-4 with BMP-5, BMP-6 or BMP-7 are strongly active in the W20 bioassay.

Heterodimers of this invention may also be characterized by activity in bone growth and stimulation assays. For example, a heterodimer of this invention is also active in the rat bone formation assay described below in Example 9. The heterodimers are also active in the osteocalcin bioassay described in Example 8. Other characteristics of a heterodimer of this invention include co-precipitation with anti-BMP antibodies to the two different constituent BMPs, as well as characteristic results on western blots, high pressure liquid chromatography (HPLC) and on two-dimensional gels, with and without reducing conditions.

One embodiment of the method of the present invention for producing recombinant BMP heterodimers involves culturing a suitable cell line, which has been co-transfected with a DNA sequence coding for expression of a first BMP or fragment thereof and a DNA sequence coding for expression of a second BMP or fragment thereof, under the control of known regulatory sequences. The transformed host cells are cultured and the heterodimeric protein recovered and purified from the culture medium.

In another embodiment of this method which is the presently preferred method of expression of the heterodimers of this invention, a single host cell, e.g., a CHO DUKX cell, is co-transfected with a first DNA molecule containing a DNA sequence encoding one BMP and a second DNA molecule containing a DNA sequence encoding a second selected BMP. One or both plasmids contain a selectable marker that can be used to establish stable cell lines expressing the BMPs. These separate plasmids containing distinct BMP genes on seperate transcription units are mixed and transfected into the CHO cells using conventional protocols. A ratio of plasmids that gives maximal expression of activity in the W20 assay, generally, 1:1, is determined.

For example, as described in detail in Example 3, equal ratios of a plasmid containing the first BMP and a dihydrofolate reductase (DHFR) marker gene and another plasmid containing a second BMP and a DHFR marker gene can be co-introduced into DHFR-deficient CHO cells, DUKX-BII, by calcium phosphate coprecipitation and transfection, electroporation, microinjection, protoplast fusion or lipofection. Individual DHFR expressing transformants are selected for growth in alpha media with dialyzed fetal calf serum by conventional means. DHFR+ cells containing increased gene copies can be selected for propagation in increasing concentrations of methotrexate (MTX) (e.g. sequential steps in 0.02, 0.1, 0.5 and 2.0 uM MTX) according to the procedures of Kaufman and Sharp, J. Mol. Biol., 159:601-629 (1982); and Kaufman et al, Mol. Cell Biol., 5:1750 (1983). Expression of the heterodimer or at least one BMP linked to DHFR should increase with increasing levels of MTX resistance. Cells that stably express either or both BMP/DHFR genes will survive. However at a high frequency, cell lines stably incorporate and express both plasmids that were present during the initial transfection. The conditioned medium is thereafter harvested and the heterodimer isolated by conventional methods and assayed for activity. This approach can be employed with DHFR-deficient cells.

As an alternative embodiment of this method, a DNA molecule containing one selected BMP gene may be transfected into a stable cell line which already expresses another selected BMP gene. For example as described in detail in Example 3 below, a stable CHO cell line expressing BMP-7 with the DHFR marker (designated 7MB9) [Genetics Institute, Inc] is transfected with a plasmid containing BMP-2 and a second selectable marker gene, e.g., neomycin resistance (Neo). After transfection, the cell is cultured and suitable cells selected by treatment with MTX and the antibiotic, G-418. Surviving cells are then screened for the expression of the heterodimer. This expression system has the advantage of permitting a single step selection.

Alternative dual selection strategies using different cell lines or different markers can also be used. For example, the use of an adenosine deaminiase (ADA) marker to amplify the second BMP gene in a stable CHO cell line expressing a different BMP with the DHFR marker may be preferable, since the level of expression can be increased using deoxycoformycin (DCF)-mediated gene amplification. (See the ADA containing plasmid described in Example 1). Alternatively, any BMP cell line made by first using this marker can then be the recipient of a second BMP expression vector containing a distinct marker and selected for dual resistance and BMP coexpression.

Still another embodiment of a method of expressing the heterodimers of this invention includes transfecting the host cell with a single DNA molecule encoding multiple genes for expression-either on a single transcription unit or on separate transcription units. Multicistronic expression involves multiple polypeptides encoded within a single transcript, which can be efficiently translated from vectors utilizing a leader sequence, e.g., from the EMC virus, from poliovirus, or from other conventional sources of leader sequences. Two BMP genes and a selectable marker can be expressed within a single transcription unit. For example, vectors containing the configuration BMPx-EMC-BMPy-DHFR or BMPx-EMC-BMPy-EMC-DHFR can be transfected into CHO cells and selected and amplified using the DHFR marker. A plasmid may be constructed which contains DNA sequences encoding two different BMPs, one or more marker genes and a suitable leader or regulatory sequence on a single transcription unit.

Similarly, host cells may be transfected with a single plasmid which contains separate transcription units for each BMP. A selectable marker, e.g., DHFR, can be contained on a another transcription unit, or alternatively as the second cistron on one or both of the BMP genes. These plasmids may be transfected into a selected host cell for expression of the heterodimer, and the heterodimer isolated from the cells or culture medium as described above.

Another embodiment of this expression method involves cell fusion. Two stable cell lines which express selected BMPs, such as a cell line expressing BMP-2 (e.g., 2EG5) and a cell line expressing BMP-7 (e.g., 7MB9), developed using the DHFR/MTX gene amplification system and expressing BMP at high levels, as described in Example 1 and in the above incorporated U.S. applications, can be transfected with one of several dominant marker genes (e.g., neon^{r}, hygromycin^{r}, GPT). After sufficient time in coculture (approximately one day) one resultant cell line expressing one BMP and a dominant marker can be fused with a cell line expressing a different BMP and preferably a different marker using a fusigenic reagent, such as polyethylene glycol, Sendai virus or other known agent.

The resulting cell hybrids expressing both dominant markers and DHFR can be selected using the appropriate culture conditions, and screened for coexpression of the BMPs or their fragments. The selected hybrid cell contains sequences encoding both selected BMPs, and the heterodimer is formed in the cell and then secreted. The heterodimer is obtained from the conditioned medium and isolated and purified therefrom by conventional methods (see e.g., Example 4). The resulting heterodimer may be characterized by methods described herein.

Cell lines generated from the approaches described above can be used to produce co-expressed, heterodimeric BMP polypeptides. The heterodimeric proteins are isolated from the cell medium in a form substantially free from other proteins with which they are co-produced as well as from other contaminants found in the host cells by conventional purification techniques. The presently preferred method of production is co-transfection of different vectors into CHO cells and methotrexate-mediated gene amplification. Stable cell lines may be used to generate conditioned media containing recombinant BMP that can be purified and assayed for in vitro and in vivo activities. For example, the resulting heterodimer-producing cell lines obtained by any of the methods described herein may be screened for activity by the assays described in Examples 8 and 9, RNA expression, and protein expression by sodium dodecyl sulfate polyacrylamide gel electrophoresis (SDS-PAGE).

The above-described methods of co-expression of the heterodimers of this invention utilize suitable host cells or cell lines. Suitable cell preferably include mammalian cells, such as Chinese hamster ovary cells (CHO). The selection of suitable mammalian host cells and methods for transformation, culture, amplification, screening and product production and purification are known in the art. See, e.g., Gething and Sambrook, Nature, 293:620-625 (1981), or alternatively, Kaufman et al, Mol. Cell. Biol., 5(7):1750-1759 (1985) or Howley et al, U.S. Pat. No. 4,419,446. Other suitable mammalian cell lines are the CV-1 cell line, BHK cell lines and the 293 cell line. The monkey COS-l cell line is presently believed to be inefficient in BMP heterodimer production.

Many strains of yeast cells known to those skilled in the art may also be available as host cells for expression of the polypeptides of the present invention, e.g., Saccharomyces cerevisiae. Additionally, where desired, insect cells may be utilized as host cells in the method of the present invention. See, e.g., Miller et al, Genetic Engineering, 8:277-298 (Plenum Press 1986) and references cited therein.

Another method for producing a biologically active heterodimeric protein of this invention may be employed where the host cells are microbial, preferably bacterial cells, in particular E. coli. For example, the various strains of E. coli (e.g., HB101, MC1061) are well-known as host cells in the field of biotechnology. Various strains of B. subtilis, Pseudomonas, other bacilli and the like may also be employed in this method.

This method, which may be employed to produce monomers and dimers (both homodimers and heterodimers) is described in European Patent Application No. 433,225, incorporated herein by reference. Briefly, this process involves culturing a microbial host comprising a nucleotide sequence encoding the desired BMP protein linked in the proper reading frame to an expression control sequence which permits expression of the protein and recovering the monomeric, soluble protein. Where the protein is insoluble in the host cells, the water-insoluble protein fraction is isolated from the host cells and the protein is solubilized. After chromatographic purification, the solubilized protein is subjected to selected conditions to obtain the biologically active dimeric configuration of the protein. This process, which may be employed to produce the heterodimers of this invention, is described specifically in Example 7, for the production of a BMP-2 homodimer.

Another aspect of the present invention provides DNA molecules or plasmid vectors for use in expression of these recombinant heterodimers. These plasmid vectors may be constructed by resort to known methods and available components known to those of skill in the art. In general, to generate a vector useful in the methods of this invention, the DNA encoding the desired BMP protein is transferred into one or more appropriate expression vectors suitable for the selected host cell.

It is presently contemplated that any expression vector suitable for efficient expression in mammalian cells may be employed to produce the recombinant heterodimers of this invention in mammalian host cells. Preferably the vectors contain the selected BMP DNA sequences described above and in the Figures, which encode selected BMP components of the heterodimer. Alternatively, vectors incorporating modified sequences as described in the above-referenced patent applications are also embodiments of the present invention and useful in the production of the vectors.

In addition to the specific vectors described in Example 1, one skilled in the art can construct mammalian expression vectors by employing the sequence of FIGS. 1-6 or other DNA sequences containing the coding sequences of FIGS. 1-6 (SEQ ID NOs: 1, 3, 5, 7, 9 and 11), or other modified sequences and known vectors, such as pCD [Okayama et al, Mol. Cell Biol., Z:161-170 (1982)] and pJL3, pJL4 [Gough et al, EMBO J., 4:645-653 (1985)]. The BMP DNA sequences can be modified by removing the non-coding nucleotides on the 5′ and 3′ ends of the coding region. The deleted non-coding nucleotides may or may not be replaced by other sequences known to be beneficial for expression. The transformation of these vectors into appropriate host cells as described above can produce desired heterodimers.

One skilled in the art could manipulate the sequences of FIGS. 1-6 by eliminating or replacing the mammalian regulatory sequences flanking the coding sequence with e.g., yeast or insect regulatory sequences, to create vectors for intracellular or extracellular expression by yeast or insect cells. [See, e.g., procedures described in published European Patent Application 155,476] for expression in insect cells; and procedures described in published PCT application WO86/00639 and European Patent Application EPA 123,289 for expression in yeast cells].

Similarly, bacterial sequences and preference codons may replace sequences in the described and exemplified mammalian vectors to create suitable expression systems for use in the production of BMP monomers in the method described above. For example, the coding sequences could be further manipulated (e.g., ligated to other known linkers or modified by deleting non-coding sequences therefrom or altering nucleotides therein by other known techniques). The modified BMP coding sequences could then be inserted into a known bacterial vector using procedures such as described in T. Taniguchi et al, Proc. Natl. Acad. Sci. USA, 77:5230-5233 (1980). The exemplary bacterial vector could then be transformed into bacterial host cells and BMP heterodimers expressed thereby. An exemplary vector for microbial, e.g., bacterial, expression is described below in Example 7.

Other vectors useful in the methods of this invention may contain multiple genes in a single transcription unit. For example, a proposed plasmid p7E2D contains the BMP-7 gene followed by the EMC leader sequence, followed by the BMP-2 gene, followed by the DHFR marker gene. Another example is plasmid p7E2ED which contains the BMP-7 gene, the EMC leader, the BMP-2 gene, another EMC leader sequence and the DHFR marker gene. Alternatively, the vector may contain more than one transcription unit. As one example, the plasmid p2ED7ED contains a transcription unit for BMP-2 and a separate transcription unit for BMP-7, i.e., BMP-2-EMC-DHFR and BMP-7-EMC-DHFR. Alternatively, each transcription unit on the plasmid may contain a different marker gene. For example, plasmid p2EN7ED contains BMP-2-EMC-Neo and BMP-7-EMC-DHFR.

Additionally the vectors also contain appropriate expression control sequences which are capable of directing the replication and expression of the BMP in the selected host cells. Useful regulatory sequences for such vectors are known to one of skill in the art and may be selected depending upon the selected host cells. Such selection is routine and does not form part of the present invention. Similarly, the vectors may contain one or more selection markers, such as the antibiotic resistance gene, Neo or selectable markers such as DHFR and ADA. The presently preferred marker gene is DHFR. These marker genes may also be selected by one of skill in the art.

Once they are expressed by one of the methods described above, the heterodimers of this invention may be identified and characterized by application of a variety of assays and procedures. A co-precipitation (immunoprecipitation) assay may be performed with antibodies to each of the BMPs forming the heterodimer. Generally antibodies for this use may be developed by conventional means, e.g., using the selected BMP, fragments thereof, or synthetic BMP peptides as antigen. Antibodies employed in assays are generally polyclonal antibodies made from individual BMP peptides or proteins injected into rabbits according to classical techniques. This assay is performed conventionally, and permits the identification of the heterodimer, which is precipitated by antibodies to both BMP components of the heterodimer. In contrast, only one of the two antibodies causes precipitation of any homodimeric form which may be produced in the process of producing the heterodimer.

Another characterizing assay is a Western assay, employing a precipitating antibody, a probing antibody and a detecting antibody. This assay may also be performed conventionally, by using an antibody to one of the BMPs to precipitate the dimers, which are run on reducing SDS-PAGE for Western analysis. An antibody to the second BMP is used to probe the-precipitates on the Western gel for the heterodimer. A detecting antibody, such as a goat-antirabbit antibody labelled with horseradish peroxidase (HRP), is then applied, which will reveal the presence of one of the component subunits of the heterodimer.

Finally, the specific activity of the heterodimer may be quantitated as described in detail in Example 6. Briefly, the amount of each BMP is quantitated using Western blot analysis or pulse labelling and SDS-PAGE analysis in samples of each BMP homodimer and the heterodimer. The W20 activity is also determined as described specifically in Example 8. The relative specific activities may be calculated by the formula: W20 alkaline phosphatase activity/amount of BMP on Western blot or by fluorography. As one example, this formula has been determined for the BMP-2/7 heterodimer, demonstrating that the heterodimer has an estimated 5 to 50 fold higher specific activity than the BMP-2 homodimer.

The heterodimers of the present invention may have a variety of therapeutic and pharmaceutical uses, e.g., in compositions for wound healing, tissue repair, and in similar compositions which have been indicated for use of the individual BMPs. Increased potency of the heterodimers over the individual BMPs may permit lower dosages of the compositions in which they are contained to be administered to a patient in comparison to dosages of compositions containing only a single BMP. A heterodimeric protein of the present invention, which induces cartilage and/or bone growth in circumstances where bone is not normally formed, has application in the healing of bone fractures and cartilage defects in humans and other animals. Such a preparation employing a heterodimeric protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A heterodimeric protein of this invention may be used in the treatment of periodontal disease, and in other tooth repair processes. Such agents may provide an environment to attract bone-forming cells, stimulate growth of bone-forming cells or induce differentiation of progenitors of bone-forming cells. Heterodimeric polypeptides of the invention may also be useful in the treatment of osteoporosis. A variety of osteogenic, cartilage-inducing and bone inducing factors have been described. See, e.g., European Patent Applications 148,155 and 169,016 for discussions thereof.

The proteins of the invention may also be used in wound healing and related tissue repair. The types of wounds include, but are not limited to burns, incisions and ulcers. (See, e.g., PCT Publication WO84/01106 incorporated by reference herein for discussion of wound healing and related tissue repair).

Additionally, the proteins of the invention may increase neuronal survival and therefore be useful in transplantation and treatment of conditions exhibiting a decrease in neuronal survival.

In view of the usefulness of the heterodimers, therefore, a further aspect of the invention is a therapeutic method and composition for repairing fractures and other conditions related to cartilage and/or bone defects or periodontal diseases. In addition, the invention comprises therapeutic methods and compositions for wound healing and tissue repair. Such compositions comprise a therapeutically effective amount of a heterodimeric protein of the invention in admixture with a pharmaceutically acceptable vehicle, carrier or matrix. The preparation and formulation of such physiologically acceptable protein compositions, having due regard to pH, isotonicity, stability and the like, is within the skill of the art.

It is expected that the proteins of the invention may act in concert with other related proteins and growth factors. Therapeutic methods and compositions of the invention therefore comprise a therapeutic amount of a heterodimeric protein of the invention with a therapeutic amount of at least one of the other BMP proteins disclosed in co-owned and concurrently filed U.S. applications described above. Such combinations may comprise separate molecules of the BMP proteins or other heteromolecules of the present invention.

In further compositions, heterodimeric proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications due to the lack of species specificity in BMP proteins. Particularly domestic animals and thoroughbred horses, in addition to humans, are desired patients for such treatment with heterodimeric proteins of the present invention.

The therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than the heterodimeric proteins of the invention which may also optionally be included in the composition as described above, may alternatively or additionally, be administered simultaneously or sequentially with the heterodimeric BMP composition in the methods of the invention. Preferably for bone and/or cartilage formation, the composition would include a matrix capable of delivering the heterodimeric protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical. properties, cosmetic appearance and interface properties. The particular application of the heterodimeric BMP compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Presently preferred is a 50:50 (mole weight) copolymer of lactic acid and glycolic acid in the form of porous particles having diameters ranging from 150 to 800 microns. In some applicatons, it will be useful to utilize a sequestering agent, such as carboxymethyl cellulose or autologous blood clot, to prevent the BMP compositions from dissassociating from the matrix.

The dosage regimen of a heterodimeric protein-containing pharmaceutical composition will be determined by the attending physician considering various factors which modify the action of the heterodimeric proteins, e.g. amount of bone weight desired to be formed, the site of bone damage, the condition of the damaged bone, the size of a wound, type of damaged tissue, the patient's age, sex, and diet, the severity of any infection, time of administration and other clinical factors. The dosage nay vary with the type of matrix used in the reconstitution and the BMP proteins in the heterodimer and any additional BMP or other proteins in the pharmaceutical composition. For example, the addition of other known growth factors, such as IGF I (insulin like growth factor I), to the final composition, may also effect the dosage. Progress can be monitored by periodic assessment of bone growth and/or repair, for example, X-rays, histomorphometric determinations and tetracycline labeling.

The following examples are illustrative of the present invention and do not limit its scope.

### EXAMPLE 1

### BMP Vector Constructs and Cell Lines

A. BMP-2 Vectors

The mammalian expression vector pMT2 CXM is a derivative of p91023 (b) [Wong et al, Science, 228:810-815 (1985)] differing from the latter in that it contains the ampicillin resistance gene (Amp) in place of the tetracycline resistance gene (Tet) and further contains a XhoI site for insertion of cDNA clones. The functional elements of pMT2 CXM have been described [R. J. Kaufman, Proc. Natl. Acad. Sci. USA, 82:689-693 (1985)] and include the adenovirus VA genes, the SV40 origin of replication including the 72 bp enhancer, the adenovirus major late promoter including a 5′ splice site and the majority of the adenovirus tripartite leader sequence present on adenovirus late mRNAs, a 3′ splice acceptor site, a DHFR insert, the SV40 early polyadenylation site (SV40), and pBR322 sequences needed for propagation in E. coli.

EcoRI digestion of pMT2-VWF, which has been deposited with the American Type Culture Collection (ATCC), Rockville, Md. (USA) under accession number ATCC 67122, excises the cDNA insert present in pMT2-VWF, yielding pMT2 in linear form. Plasmid pMT2 can be ligated and used to transform E. coli HB 101 or DH-5 to ampicillin resistance. Plasmid pMT2 DNA can be prepared by conventional methods.

Plasmid pMT2 CXM is then constructed using loopout/in mutagenesis [Morinaga et al, Biotechnology, 84:636 (1984)]. This removes bases 1075 to 1145 relative to the HindIII site near the SV40 origin of replication and enhancer sequences of pMT2. In addition it inserts the following sequence:

5′ PO₄-CATGGGCAGCTCGAG-3′ (SEQ ID No: 15) at nucleotide 1145. This sequence contains the recognition site for the restriction endonuclease XhoI.

A derivative of pMT2 CXM, termed plasmid pMT23, contains recognition sites for the restriction endonucleases PstI, EcoRI, SalI and XhoI.

Full length BMP-2 cDNA (FIG. 1) (SEQ ID NO: 1) is released from the λGT10 vector by digestion with EcoRI and subcloned into pSP65 [Promega Biotec, Madison, Wis.; see, e.g., Melton et al, Nucl. Acids Res., 12:7035-7056 (1984)] in both orientations yielding pBMP-2 #39-3 or pBMP-2 #39-4.

The majority of the untranslated regions of the BMP-2 cDNA are removed in the following manner. The 5′ sequences are removed between the SalI site in the adapter (present from the original cDNA cloning) and the SalI site 7 base pairs upstream of the initiator ATG by digestion of the pSP65 plasmid containing the BMP-2 cDNA with SalI and religation. The 3′ untranslated region is removed using heteroduplex mutagenesis using the oligonucleotide
5 GAGGGTTGTGGGTGTCGCTAGTGAGTCGACTACAGCAAAATT3′. End SalI (SEQ ID NO: 16)
   The sequence contains the terminal 3′ coding region of the BMP-2 cDNA, followed immediately by a recognition site for SalI. The sequence introduces a SalI site following the termination (TAG) codon.

The SalI fragment of this clone was subcloned into the expression vector pMT23, yielding the vector pMT23-BMP2ΔUT. Restriction enzyme sites flank the BMP-2 coding region in the sequence PstI-EcoRI-SalI-BMP-2 cDNA-SalI-EcoRI-XhoI.

The expression plasmid pED4 [Kaufman et al, Nucl. Acids Res., 19:4485-4490 (1991)] was linearized by digestion with EcoRI and treated with calf intestinal phosphatase. The BMP-2 cDNA gene was excised from pMT23-BMP2ΔUT by digestion with EcoRI and recovery of the 1.2 kb fragment by electrophoresis through a 1.0% low melt agarose gel. The linearized pED4 vector and the EcoRI BMP-2 fragment were ligated together, yielding the BMP-2 expression plasmid pBMP2Δ-EMC.

Another vector pBMP-2Δ-EN contains the same sequences contained within the vector pBMP2Δ-EMC, except the DHFR gene has been replaced by conventional means with the neomycin resistance gene from the Tn5 transposable element.

B. BMP4 Vectors

A BMP-4 cDNA sequence set forth in FIG. 2 (SEQ ID NO: 3), in which the 3′ untranslated region is removed, is made via heteroduplex mutagenesis with the mutagenic oligonucleotide:

(SEQ ID NO:17)

5′ GGATGTGGGTGCCGCTGACTCTAGAGTCGACGGAATTC 3′
End EcoRI

This deletes all of the sequences 3′ to the translation terminator codon of the BMP-4 cDNA, juxtaposing this terminator codon and the vector polylinker sequences. This step is performed in an SP65 vector [Promega Biotech] and may also be conveniently performed in pMT2-derivatives containing the BMP-4 cDNA similar to the BMP2 vectors described above. The 5′ untranslated region is removed using the restriction endonuclease BsmI, which cleaves within the eighth codon of BMP-4 cDNA.

Reconstruction of the first eight codons is accomplished by ligation to oligonucleotides:

EcoRI Initiator BsmI
5′ AATTCAACCATGATTCCTGGTAACCGAATGCT 3′ (SEQ ID NO: 18) and
3′ GTGGTACTAAGGACCATTGGCTTAC 5′ (SEQ ID NO: 19)
   These oligonucleotides form a duplex which has a BsmI complementary cohesive end capable of ligation to the BsmI restricted BMP-4 cDNA, and it has an EcoRI complementary cohesive end capable of ligation to the EcoRI restricted vector pMT2. Thus the cDNA for BMP-4 with the 5′ and 3′ untranslated regions deleted, and retaining the entire encoding sequence is contained within an EcoRI restriction fragment of approximately 1.2 kb.

The pNT2 CXM plasmid containing this BMP-4 sequence is designated pXMBMP-4ΔUT. It is digested with EcoRI in order to release the BMP-4 cDNA containing insert from the vector. This insert is subcloned into the EcoRI site of the mammalian expression vector pED4, resulting PBMP4A-EMC.

C. BMP-5 Vectors

A BMP-5 cDNA sequence comprising the nucleotide sequence from nucleotide #699 to #2070 of FIG. 5 (SEQ ID NO: 9) is specifically amplified as follows. The oligonucleotides CGACCTGCAGCCACCATGCATCTGACTGTA (SEQ ID NO: 20) and TGCCTGCAGTTTAATATTAGTGGCAGC (SEQ ID NO: 21) are utilized as primers to allow the amplification of nucleotide sequence #699 to #2070 of FIG. 5 from the BMP-5 insert of λ-ZAP clone U2-16 [ATCC #68109]. This procedure introduces the nucleotide sequence CGACCTGCAGCCACC (SEQ ID NO: 22) immediately preceeding nucleotide #699 and the nucleotide sequence CTGCAGGCA immediately following nucleotide #2070. The addition of these sequences results in the creation of PstI restriction endonuclease recognition sites at both ends of the amplified DNA fragment. The resulting amplified DNA product of this procedure is digested with the restriction endonuclease PstI and subcloned into the PstI site of the pMT2 derivative pMT21 [Kaufman, Nucl. Acids Res., 19:4485-4490 (1991)]. The resulting clone is designated H5/5/pMT.

The insert of H5/5/pMT is excised by PstI digestion and subcloned into the plasmid vector pSP65 [Promega Biotech] at the PstI site, resulting in plasmid BMP5/SP6. BMP5/SP6 and U2-16 are digested with the restriction endonucleases NsiI and NdeI to excise the portion of their inserts corresponding to nucleotides #704 to #1876 of FIG. 5. The resulting 1173 nucleotide NsiI-NdeI fragment of clone U2-16 is ligated into the NsiI-NdeI site of BMP5/SP6 from which the corresponding 1173 nucleotide NsiI-NdeI fragment had been removed. The resulting clone is designated BMP5mix/SP65.

Direct DNA sequence analysis of BMPSmix/SP65 is performed to confirm identity of the nucleotide sequences produced by the amplification to those set forth in FIG. 5. The clone BMP5mix/SP65 is digested with the restriction endonuclease PstI resulting in the excision of an insert comprising the nucleotides #699 to #2070 of FIG. 5 and the additional sequences containing the PstI recognition sites as described above. The resulting 1382 nucleotide PstI fragment is subcloned into the PstI site of the pMT2 derivative pMT21. This clone is designated BMP5mix/pMT21#2.

The same fragment is also subloned into the PstI site of pED4 to yield the vector designated BMP5mix-EMC-11.

D. BMP-6 Vectors

A BMP-6 cDNA sequence comprising the nucleotide sequence from nucleotide #160 to #1706 of FIG. 4 (SEQ ID No: 7) is produced by a series of techniques known to those skilled in the art. The clone BMP6C35 [ATCC 68245] is digested with the restriction endonucleases ApaI and TaqI, resulting in the excision of a 1476 nucleotide portion of the insert comprising nucleotide #231 to #1703 of FIG. 4. Synthetic oligonucleotides with SalI restriction endonuclease site converters are designed to replace those nucleotides corresponding to #160 to #230 and #1704 to #1706 which are not contained in the 1476 ApaI-TaqI fragment of the BMP-6 cDNA sequence.

Oligonucleotide/SalI converters conceived to replace the missing 5′ (TCGACCCACCATGCCGGGGCTGGGGCGGAGGGCGCAGTGGCTGT GCTGGTGGTGGGGGCTGTGCTGCAGCTGCTGCGGGCC (SEQ ID NO: 23) and CGCAGCAGCTGCACAGCAGCCCCCACCACCAGCACAGCCACTGCGCCCTCCGCCCCA GCCCCGGCATGGTGGG) (SEQ ID NO: 24) and 3′ (TCGACTGGTTT (SEQ ID NO: 25) and CGAAACCAG (SEQ ID NO: 26) ) sequences are annealed to each other independently. The annealed 5′ and 3′ converters are then ligated to the 1476 nucleotide Apal-TaqI described above, creating a 1563 nucleotide fragment comprising the nucleotide sequence from #160 to #1706 of FIG. 4 and the additional sequences contrived to create SalI restriction endonuclease sites at both ends. The resulting 1563 nucleotide fragment is subcloned into the SalI site of pSP64 [Promega Biotech, Madison, Wis.]. This clone is designated BMP6/SP64#15.

DNA sequence analysis of BMP6/SP64#15 is performed to confirm identity of the 5′ and 3′ sequences replaced by the converters to the sequence set forth in FIG. 4. The insert of BMP6/SP64#15 is excised by digestion with the restriction endonuclease SalI. The resulting 1563 nucleotide SalI fragment is subcloned into the XhoI restriction endonuclease site of pMT21 and designated herein as BMP6/pMT21.

The PstI site of pED4 is converted to a SalI site by digestion of the plasmid with PstI and ligation to the converter oligonucleotides:
5′-TCGACAGGCTCGCCTGCA-3′ (SEQ ID NO: 27) and
3′-GTCCGAGCGG-5′ (SEQ ID NO: 28).
   The above 1563 nucleotide SalI fragment is also subcloned into the SalI site of this pED4 vector, yielding the expression vector BMP6/EMC.
   E. BMP-7 Vectors

A BMP-7 sequence comprising the nucleotide sequence from nucleotide #97 to #402 of FIG. 3 (SEQ ID NO: 5) is specifically amplified as follows. The oligonucleotides CAGGTCGACCCACCATGCACGTGCGCTCA (SEQ ID NO: 29) and TCTGTCGACCTCGGAGGAGCTAGTGGC (SEQ ID NO: 30) are utilized as primers to allow the amplification of nucleotide sequence #97 to #1402 of FIG. 3 from the insert of clone PEH7-9 [ATCC #68182]. This procedure generates the insertion of the nucleotide sequence CAGGTCGACCCACC immediately preceeding nucleotide #97 and the insertion of the nucleotide sequence GTCGACAGA immediately following nucleotide #1402. The addition of these sequences results in the creation of a SalI restriction endonuclease recognition site at each end of the amplified DNA fragment. The resulting amplified DNA product of this procedure is digested with the restriction endonuclease SalI and subcloned into the SalI site of the plasmid vector pSP64 (Promega Biotech, Madison, Wis.). resulting in BMP7/SP6#2.

The clones BMP7/SP6#2 and PEH7-9 are digested with the restriction endonucleases NcoI and StuI to excise the portion of their inserts corresponding to nucleotides #363 to #1081 of FIG. 3. The resulting 719 nucleotide NcoI-StuI fragment of clone PEH7-9 is ligated into the NcoI-StuI site of BMP7/SP6#2 from which the corresponding 719 nucleotide fragment is removed. The resulting clone is designated BMP7mix/SP6.

Direct DNA sequence analysis of BMP7mix/SP6 confirmed identity of the 3′ region to the nucleotide sequence from #1082 to #1402 of FIG. 3, however the 5′ region contained one nucleotide misincorporation.

Amplification of the nucleotide sequence (#97 to #1402 of FIG. 3) utilizing PEH7-9 as a template is repeated as described above. The resulting amplified DNA product of this procedure is digested with the restriction endonucleases SalI and PstI. This digestion results in the excision of a 747 nucleotide fragment comprising nucleotide #97 to #833 of FIG. 3 plus the additional sequences of the 5′ priming oligonucleotide used to create the SalI restriction endonuclease recognition site described earlier. This 747 SalI-PstI fragment is subcloned into a SalI-PstI digested pSP65 [Promega Biotech, Madison, Wis.] vector resulting in 5′BMP7/SP65. DNA sequence analysis demonstrates that the insert of the 5′BMP7/SP65#1 comprises a sequence identical to nucleotide #97 to #362 of FIG. 3.

The clones BMP7mix/SP6 and 5′BMP7/SP65 are digested with the restriction endonucleases SalI and NcoI. The resulting 3′ NcoI-SalI fragment of BMP7mix/SP6 comprising nucleotides #363 to #1402 of FIG. 3 and 5′ SalI-NcoI fragment of 5′BMP7/SP65 comprising nucleotides #97 to #362 of FIG. 3 are ligated together at the NcoI restriction sites to produce a 1317 nucleotide fragment comprising nucleotides #97 to #1402 of FIG. 3 plus the additional sequences derived from the 5′ and 3′ oligonucleotide primers which allows the creation of SalI restriction sites at both ends of this fragment.

This 1317 nucleotide SalI fragment is ligated into the SalI site of the pMT2 derivative pMT2Cla-2. pMT2Cla-2 is constructed by digesting pMT21 with EcoRV and XhoI, treating the digested DNA with Klenow fragment of DNA polymerase I and ligating ClaI linkers (NEBio Labs, CATCGATG). This removes bases 2171 to 2420 starting from the HindIII site near the SV40 origin of replication and enhancer sequences of pMT2 and introduces a unique ClaI site, but leaves the adenovirus VAI gene intact, resulting in pMT2Cla-2. This clone is designated BMP-7-pMT2.

The insert of BMP-7-pMT2 is excised by digestion with the restriction endonuclease SalI. The resulting 1317 nucleotide SalI fragment is subcloned into the XhoI restriction endonuclease site of pMT21 to yield the clone BMP-7/pMT21. This SalI fragment is also subcloned into the SalI site of the pED4 vector in which the PstI site was converted into a SalI site as described above, resulting in the vector pBMP7/EMC#4.

F. BMP-8 Vectors

At present no mammalian BMP-8 vectors have been constructed. However, using the sequence of FIG. 6 (SEQ ID NO: 11), it is contemplated that vectors similar to those described above for the other BMPs may be readily constructed. A bacterial expression vector similar to the BMP-2 vector described in detail in Example 7 may also be constructed for BMP-8, by introducing a Met before the amino acid #284 Ala of FIG. 6. This sequence of BMP-8 is inserted into the vector pALBP2-781 in place of the BMP-2 sequence. See Example 7.

G. BMP Vectors Containing the Adenosine Deaminase (Ada) Marker

BMP genes were inserted into the vector pMT3SV2Ada [R. J. Kaufman, Meth. Enz., 185:537-566 (1990)] to yield expression plasmids containing separate transcription units for the BMP cDNA gene and the selectable marker Ada. pMT3SV2Ada contains a polylinker with recognition sites for the enzymes PstI, EcoRI, SalI and XbaI that can be used for insertion of and expression of genes (i.e. BMP) in mammalian cells. In addition, the vector contains a second transcription unit encoding Ada which serves as a dominant and amplifiable marker in mammalian cells.

To construct expression vectors for BMP-5, BMP-6 and BMP-7, individually, the same general method was employed. The gene for BMP 5 (FIG. 5), 6 (FIG. 4) or 7 (FIG. 3) was inserted into the polylinker essentially as described above for the pED4 vector. These vectors can be used for transfection into CHO DUKX cells and subsequent selection and amplification using the Ada marker as previously described [Kaufman et al, Proc. Natl. Acad. Sci. USA, 83:3136-3140 (1986)]. Since each such vector does not contain a DHFR gene, the resultant transformed cells remain DHFR negative and can be subsequently transfected with a second vector containing a different BMP in conjunction with DHFR and amplified with methotrexate.

Alternatively, the pMT3SV2Ada/BMP vectors can be used to transfect stable CHO cell lines previously transfected with a different BMP gene and amplified using the DHFR/methotrexate system. The resultant transfectants can be subsequently amplified using the Ada system, yielding cell lines that coexpress two different BMP genes, and are amplified using both the DHFR and Ada markers.

H. BMP-Expressing Mammalian Cell Lines

At present, the most desirable mammalian cell lines for use in producing the recombinant homodimers and heterodimers of this invention are the following. These cell lines were prepared by conventional transformation of CHO cells using vectors described above.

The BMP-2 expressing cell line 2EG5 is a CHO cell stably transformed with the vector pBMP2delta-EMC.

The BMP-4 expressing cell line 4E9 is a CHO cell stably transformed with the vector pBMP4delta-EMC.

The BMP-5 expressing cell line 5E10 is a CHO cell stably transformed with the vector BMP5mix-EMC-11 (at a amplification level of 2 micromolar MTX).

The BMP-6 expressing cell line 6HG8 is a CHO cell stably transformed with the vector BMP6/EMC.

The BMP-7 expressing cell line 7MB9 is a CHO cell stably transformed with the vector BMP7/pMT21.

### EXAMPLE 2

### Transient Expression of BMP Heterodimers

The heterodimers of the present invention may be prepared by co-expression in a transient expression system for screening in the assays of Example 8 by two different techniques as follows.

In the first procedure, the pMT2-derived and EMC-derived expression plasmids described in Example 1 and other similarly derived vectors were constructed which encoded, individually, BMP-2 through BMP-7, and transforming growth factor-beta (TGFβ1). All combinations of pairs of plasmids were mixed in equal proportion and used to co-transfect CHO cells using the DEAE-dextran procedure [Sompayrac and Danna, Proc. Natl. Acad. Sci. USA, 78:7575-7578 (1981); Luthman and Magnusson, Nucl. Acids Res., 11:1295-1308 (1983)]. The cells are grown in alpha Minimal Essential Medium (α-MEM) supplemented with 10% fetal bovine serum, adenosine, deoxyadenosine, thymidine (100 μg/ml each), pen/strep, and glutamine (1 mM).

The addition of compounds such as heparin, suramin and dextran sulfate are desirable in growth medium to increase the amounts of BMP-2 present in the conditioned medium of CHO cells. Similarly responsive to such compounds is BMP-5. Therefore, it is expected that these compounds will be added to growth medium for any heterodimer containing these BMP components. Other BMPs may also be responsive to the effects of these compounds, which are believed to inhibit the interaction of the mature BMP molecules with the cell surface.

The following day, fresh growth medium, with or without 100 μg/ml heparin, was added. Twenty-four hours later, conditioned medium was harvested.

In some experiments, the conditioned medium was collected minus heparin for the 24-48 hour period post-transfection, and the same plates were then used to generate conditioned medium in the presence of heparin 48-72 hour post-transfection. Controls included transfecting cells with expression plasmids lacking any BMP sequences, transfecting cells with plasmids containing sequences for only a single BMP, or mixing conditioned medium from cells transfected with a single BMP with conditioned medium from cells transfected with a different BMP.

Characterizations of the coexpressed heterodimer BMPs in crude conditioned media, which is otherwise not purified, provided the following results. Transiently coexpressed BMP was assayed for induction of alkaline phosphatase activity on W20 stromal cells, as described in Example 8.

Co-expression of BMP-2 with BMP-5, BMP-6 and BMP-7, and BMP-4 with BMP-5, BMP-6 and BMP-7 yielded more alkaline phosphatase inducing activity in the W20 assay than either of the individual BMP homodimers alone or mixtures of homodimers, as shown below. Maximal activity (in vitro), was obtained when BMP-2 was coexpressed with BMP-7. Increased activity was also found the heterodimers BMP-2/5; BMP-2/6; BMP-4/5; BMP-4/6; and BMP-4/7.

| TGF- | BMP- | BMP- | BMP- | BMP- | BMP- | BMP- |
|---|---|---|---|---|---|---|
| β | 7 | 6 | 5 | 4 | 3 | 2 |

Condition Medium

| | | | | | | | |
|---|---|---|---|---|---|---|---|
| BMP-2 | 33 | 240 | 99 | 89 | 53 | 9 | 29 |
| BMP-3 | — | — | — | — | 14 | — | |
| BMP-4 | 12 | 115 | 25 | 22 | 24 | | |
| BMP-5 | — | — | — | — | | | |
| BMP-6 | — | — | — | | | | |
| BMP-7 | — | — | | | | | |
| TGF-β | — | | | | | | |

Condition Medium + heparin

| | | | | | | | |
|---|---|---|---|---|---|---|---|
| BMP-2 | 88 | 454 | 132 | 127 | 70 | 77 | 169 |
| BMP-3 | — | — | — | — | 7 | — | |
| BMP-4 | 7 | 119 | 30 | 41 | 37 | | |
| BMP-5 | — | — | — | — | | | |
| BMP-6 | — | — | — | | | | |
| BMP-7 | — | — | | | | | |
| TGF-β | — | | | | | | |

Units: 1 unit of activity is equivalent to that of 1 ng/ml of rhBMP-2.
—: indicates activity below the detection limit of the assay.

These BMP combinations were subsequently expressed using various ratios of expression plasmids (9:1, 3:1, 1:1, 1:3, 1:9) during the CHO cell transient transfection. The performance of this method using plasmids containing BMP-2 and plasmids containing BMP-7 at plasmid number ratios ranging from 9:1 to 1:9, respectively, demonstrated that the highest activity in the W20 assay was obtained when approximately the same number of plasmids of each BMP were transfected into the host cell. Ratios of BMP-2 to BMP-7 plasmids of 3:1 to 1:3, respectively, also resulted in increased activity in W20 assay in comparison to host cells transfected with plasmids containing only a single BMP. However, these latter ratios produced less activity than the 1:1 ratio.

Similar ratios may be determined by one of skill in the art for heterodimers consisting of other than BMP-2 and BMP-7. For example, preliminary work on the heterodimer formed between BMP-2 and BMP-6 has indicated that a preferred ratio of plasmids for co-transfection is 3:1, respectively. The determination of preferred ratios for this method is within the skill of the art.

As an alternative means to transiently generate coexpressed BMPs, the stable CHO cell lines identified in Example 1 expressing each BMP-2, BMP-4, BMP-5, BNP-6 and BMP-7, are cocultured for one day, and are then fused with 46.7% polyethylene glycol (PEG). One day post-fusion, fresh medium is added and the heterodimers are harvested 24 hours later for the W20 assay, described in Example 8. The assay results were substantially similar to those described immediately above.

Therefore, all combinations of BMP-2 or 4 coexpressed with either BMP-5, 6 or 7 yielded greater activity than any of the BMP homodimers alone. In control experiments where each BMP homodimer was expressed alone and conditioned media mixed post harvest, the activity was always intermediate between the individual BMPs, demonstrating that the BMP co-expressed heterodimers yield higher activity than combinations of the individually expressed BMP homodimers.

### EXAMPLE 3

### Stable Expression of BMP Heterodimers

A. BMP-2/7

Based on the results of the transient assays in Example 2, stable cell lines were made that co-express BMP-2 and BMP-7.

A preferred stable cell line, 2E7E-10, was obtained as follows: Plasmid DNA (a 1:1 mixture of pBMP-7-EMC and pBMP-2-EMC, described in Example 1) is transfected into CHO cells by electroporation [Neuman et al, EMBO J., 1:841-845 (1982)].

Two days later, cells are switched to selective medium containing 10% dialyzed fetal bovine serum and lacking nucleosides. Colonies expressing DHFR are counted 10-14 days later. Individual colonies or pools of colonies are expanded and analyzed for expression of each heterodimer BMP component RNA and protein using standard procedures and are subsequently selected for amplification by growth in increasing concentrations of MTX. Stepwise selection of the preferred clone, termed 2E7E, is carried out up to a concentration of 0.5 μM MTX. The cell line is then subcloned and assayed for heterodimer 2/7 expression.

Procedures for such assay include Western blot analysis to detect the presence of the component DNA, protein analysis and SDS-PAGE analysis of metabolically labelled protein, W20 assay, and analysis for cartilage and/or bone formation activity using the ectopic rat bone formation assay of Example 9. The presently preferred clonally-derived cell line is identified as 2E7E-10. This cell line secretes BMP-2/7 heterodimer proteins into the media containing 0.5 μM MTX.

The CHO cell line 2E7E-10 is grown in Dulbecco's modified Eagle's medium (DMEM)/Ham's nutrient mixture F-12, 1:1 (vol/vol), supplemented with 10% fetal bovine serum. When the cells are 80 to 100% confluent, the medium is replaced with serum-free DMEM/F-12. Medium is harvested every 24 hours for 4 days. For protein production and purification the cells are cultured serum-free.

While the co-expressing cell line 2E7E-10 preliminarily appears to make lower amounts of BMP protein than the BMP2-expressing cell line 2EG5 described in Example 2, preliminary evidence suggests that the specific activity of the presumptive heterodimer is at least 5-fold greater than BMP-2 homodimer (see Example 6).

To construct another heterodimer producing cell line, the stable CHO cell line 7MB9, previously transfected with pBMP-7-pMT2, and which expresses BMP-7, is employed. 7MB9 may be amplified and selected to 2 μM methotrexate resistance using the DHFR/MTX system. To generate a stable co-expressing cell line, cell line 7MB9 is transfected with the expression vector pBMP-2Δ-EN (EMC-Neo) containing BMP-2 and the neomycin resistance gene from the Tn5 transposable element. The resulting transfected stable cell line was selected for both G-418 and MTX resistance. Individual clones were picked and analyzed for BMP expression, as described above.

It is anticipated that stable cell lines co-expressing other combinations of BMPs which show enhanced activity by transient coexpression will likewise yield greater activity upon stable expression.

B. BMP-2/6

Based on the results of the transient assays in Example 2, stable cell lines were made that co-express BMP-2 and BMP-6.

A preferred stable cell line, 12C07, was obtained as follows: Plasmid DNA (a 1:3 mixture of pBMP-6-EMC and pBMP-2-EMC, described in Example 1) is transfected into CHO cells by electroporation [Neuman et al, EMBO J., 1:841-845 (1982)].

Two days later, cells are switched to selective medium containing 10% dialyzed fetal bovine serum and lacking nucleosides. Colonies expressing DHFR are counted 10-14 days later. Individual colonies or pools of colonies are expanded and analyzed for expression of each heterodimer BMP component RNA and protein using standard procedures and are subsequently selected for amplification by growth in increasing concentrations of MTX. Stepwise selection of the preferred clone, termed 12-C, is carried out up to a concentration of 2.0 μM MTX. The cell line is then subcloned and assayed for heterodimer 2/6 expression.

Procedures for such assay include Western blot analysis to detect the presence of the component DNA, protein analysis and SDS-PAGE analysis of metabolically labelled protein, W20 assay, and analysis for cartilage and/or bone formation activity using the ectopic rat bone formation assay of Example 9. The presently preferred clonally-derived cell line is identified as 12C07. This cell line secretes BMP-2/6 heterodimer proteins into the media containing 2.0 μM MTX.

The CHO cell line 12C07 is grown in Dulbecco's modified Eagle's medium (DMEM)/Ham's nutrient mixture F-12, 1:1 (vol/vol), supplemented with 10% fetal bovine serum. When the cells are 80 to 100% confluent, the medium is replaced with serum-free DMEM/F-12. Medium is harvested every 24 hours for 4 days. For protein production and purification the cells are cultured serum-free.

While the co-expressing cell line 12C07 preliminarily appears to make lower amounts of BMP protein than the BMP2-expressing cell line 2EG5 described in Example 2, preliminary evidence suggests that the specific activity of the presumptive heterodimer is at least 3-5-fold greater than BMP-2 homodimer (see Example 6).

To construct another heterodimer producing cell line, the stable CHO cell line 2EG5, previously transfected with pBMP-2-EMC, and which expresses BMP-2, is employed. 2EG5 may be amplified and selected to 2 μM methotrexate resistance using the DHFR/MTX system. To generate a stable co-expressing cell line, cell line 2EG5 is transfected with the expression vector pBMP-6-ada (ada deaminase) containing BMP-6 and the ADA resistance gene. The resulting transfected stable cell line was selected for both DCF and MTX resistance. Individual clones are picked and analyzed for BMP expression, as described above.

It is anticipated that stable cell lines co-expressing other combinations of BMPs which show enhanced activity by transient coexpression will likewise yield greater activity upon stable expression.

### EXAMPLE 4

### Purification of BMP2/7 and BMP-2/6 Heterodimer

The same purification procedure is used for BMP-2/6 heterodimer and BMP-2/7 heterodimer. Conditioned media from cultures of cell line 2E7E-10 or 12C07 containing recombinantly produced BMP heterodimer 2/7V or 2/6, respectively, can be generated from either adherent or suspension cultures. For small to medium scale generation of coexpressed BMP, adherent cultures are seeded into roller bottles and allowed to grow to confluence in alpha-Minimal Eagles Medium [α-MEM, Gibco, Grand Island, N.Y.] containing 10% dialyzed heat-inactivated fetal calf serum [Hazleton, Denver, Pa.]. The media is then switched to a serum-free, albumin free, low protein medium based on a 50:50 mixture of Delbecco's Modified Eagle's medium and Hams F-12 medium, optionally supplemented with 100 micrograms/ml dextran sulfate. Four or five daily harvests are pooled, and used to purify the recombinant protein.

Conditioned medium from roller bottle cultures obtained as described above was thawed slowly at room temperature and pooled. The pH of the pooled medium was adjusted to pH 8.0 using 1 M Tris, pH 8.0. A column was poured containing Matrex Cellufine Sulfate [Amicon] and equilibrated in 50 mM Tris, pH 8.0.

Upon completion of loading of the medium, the column was washed with buffer containing 50 mM Tris, 0.4 M NaCl, pH 8.0 until the absorbance at 280 nm reached baseline. The column was then washed with 50 mM Tris, pH 8.0 to remove NaCl from the buffer. The resin was then washed with 50 mM Tris, 0.2 M NaCl, 4 M Urea, pH 8.0 until a peak had eluted. The column was then washed into 50 mM Tris, pH 8.0 to remove the urea.

The bound BMP-2/7 or BMP-2/6 was then eluted using 50 mM Tris, 0.5 M NaCl, 0.5 M Arginine, pH 8.0. The eluate was collected as a single pool and may be optionally stored frozen prior to further purification. This Cellufine Sulfate eluate was diluted with 14 volumes of 6M urea and the pH of the sample was then adjusted to 6.0. A hydroxyapatite-Ultrogel [IBF] column was poured and equilibrated with 80 mM potassium phosphate, 6M urea, pH 6.0.

After the completion of sample loading, the column was washed with 10 bed volumes of the equilibration buffer. Bound BMP-2/7 or BMP-2/6 heterodimers were eluted with 5 bed volumes of 100 mM potassium phosphate, 6M urea, pH 7.4. This eluate was loaded directly onto a Vydac C₄ reverse-phase HPLC column equilibrated in water—0.1% TFA. BMP-2/7 or BMP-2/6 heterodimers were eluted with a gradient of 30-50% acetonitrile in water—0.1% trifluoroacetic acid.

Fractions containing BMPs are identified by SDS-PAGE in the presence or absence of reductant. The identity of the BMPs with respect to the heterodimers vs. homodimers is determined by 2D-PAGE (± reductant). Fractions with heterodimers gave bands which reduce to two spots. Bands from homodimer fractions reduce to a single spot for each BMP species.

The BMP-2/6 heterodimer subunits are analyzed on a protein sequenator. BMP-2/6 heterodimers of the following species are present: BMP-6 subunit beginning with amino acid #375 Ser-Ala-Ser-Ser in association with BMP-2 subunit beginning with amino acid #283 Gin-Ala-Lys or #249 Ser-Lev-His, though other less abundant species may be present. It is contemplated that the same or substantially similar purification techniques may be employed for any recombinant BMP heterodimer of this invention. The hydroxyapatite-Ultrogel column may be unnecessary and that the purification scheme may be modified by loading the Cellufine Sulfate eluate directly onto the C₄ reverse-phase HPLC column without use of the former column for BMP2/7 or BMP-2/6 or the other heterodimers of this invention.

### Example 5

### Protein Characterization

Total protein secreted from the co-expressing cell lines is analyzed after labelling with ³⁵S-methionine or by Western blot analysis using antibodies raised against both BMPs of the heterodimer, e.g., BMP-2 and BMP-7. Together with the alkaline phosphatase assays, the data indicates the presence of the heterodimer and the specific activity. The following specific details are directed towards data collected for the BMP-2/7 and BMP-2/6 heterodimers; however, by application of similar methods to the other heterodimers described herein, similar results are expected.

A. ³⁵S-Met Labelling

Cell lines derived by cotransfection of BMP2_{Δ}-EMC and BMP7_{Δ}-EMC expression vectors were pulsed with ³⁵S-methionine for 15 minutes, and chased for 6 hours in serum free media in the presence or absence of heparin. Total secreted protein was analyzed under reducing conditions by PAGE and fluorography. The results demonstrate that several cell lines secrete both BMP-2 and BMP-7 protein. There is a good correlation between the amount of alkaline phosphatase activity and the amount of coexpressed protein.

Several cell lines secrete less total BMP-2 and 7 than the BMP-2-only expressing cell line 2EG5, which produces 10 μg/ml BMP-2. Cell line 2E7E-10 (amplified at a level of 0.5 mM MTX) secretes equal proportions of BMP-2 and BMP-7 at about the same overall level of expression as the cell line 2EG5. Cell line 2E7E-10 produces the equivalent of 600 micrograms/ml of BMP-2 homodimer activity in one assay.

Total labelled protein was also analyzed on a two-dimensional non-reducing/reducing gel system to ascertain whether a heterodimer is made. Preliminary results demonstrate the presence of a unique spot in this gel system that is not found in either the BMP-2-only or BMP-7-only cell lines, suggesting the presence of 2/7 heterodimer. The same gel with purified material produced the same results (e.g., two unique spots on the gel) indicative of the presence of the 2/7 heterodimer. The homodimer of BMP2 produced distinct species on this gel system.

In contrast to the recombinant BMP-2/7 purification, BMP-2 homodimers are not detected during the BMP-2/6 preparation; however, significant amounts of BMP-6 homodimers are found. In addition, a significant amount of a −20 amino acid N-terminal truncated form of BMP-6 is found; this could be eliminated by the inclusion of protease inhibitors during cell culture. BMP-2/6 was found to elute two to three fractions later from C4 RP-HPLC than did BMP-2/7.

B. Immunoprecipitation Coupled to Western Blot Analysis

Conditioned media from a BMP-2-only (2EG5), a BMP-7-only (7MB9), or the 2E7E-10 co-expressing cell line were subjected to immunoprecipitation with either a BMP-2 or BMP-7 antibody (both conventional polyclonal antibodies raised in rabbits), then analyzed on Western blots probed with either an anti-BMP-2 or anti-BMP-7 antibody. The 2/7 heterodimer precipitates and is reactive on Western blots with both the BMP-2 and BMP-7 antibodies, while either BMP by itself reacts with its specific antibody, but not with the reciprocal antibody.

It has been demonstrated using this strategy that a protein in the co-expressing cell line that is precipitated by the anti-BMP-7 antibody W33 [Genetics Institute, Inc, Cambridge, Mass.] and reacts on a Western blot with the anti-BMP-2 antibody W12 or W10 [Genetics Institute, Inc.] is not present in the BMP-2 or 7-only expressing cell lines. This experiment indicates that this protein species is the heterodimeric protein. Conversely, precipitation with W12 and probing with W33 yielded similar results.

### EXAMPLE 6

### Specific Activity of Heterodimers

A. In vitro Assays

The specific activity of the BMP-2/7 or BMP-2/6 heterodimer and the BMP-2 homodimer secreted into growth medium of the stable cell lines 2E7E-10 and 2EG55, and 12C07 and 2EG5, respectively, were estimated as follows.

The amount of BMP protein in conditioned medium was measured by either Western blot analysis or by analyzing protein secreted from ³⁵S-methionine labelled cells by PAGE and fluorography. The amount of activity produced by the same cell lines on W20 cells using either the alkaline phosphatase assay or osteocalcin-induction assay was then estimated. The specific activity, of the BMP was calculated from the ratio of activity to protein secreted into the growth medium.

In one experiment 2E7E-10 and 2EG5 secreted similar amounts of total BMP proteins as determined by PAGE and fluorography. 2E7E-10 produced about 50-fold more alkaline phosphatase inducing activity the 2EG5, suggesting that the specific activity of the heterodimer is about 50-fold higher than the homodimer.

In another experiment the amount of BMP-2 secreted by 2EG5 was about 50% higher than BMP-2/7 secreted by 2E7E-10, however, 2E7E-10 produced about 10-fold more osteocalcin-inducing activity that 2EG5. From several different experiments of this type the specific activity of the BMP-2/7 heterodimer is estimated to be between 5 to 50 fold higher than the BMP-2 homodimer.

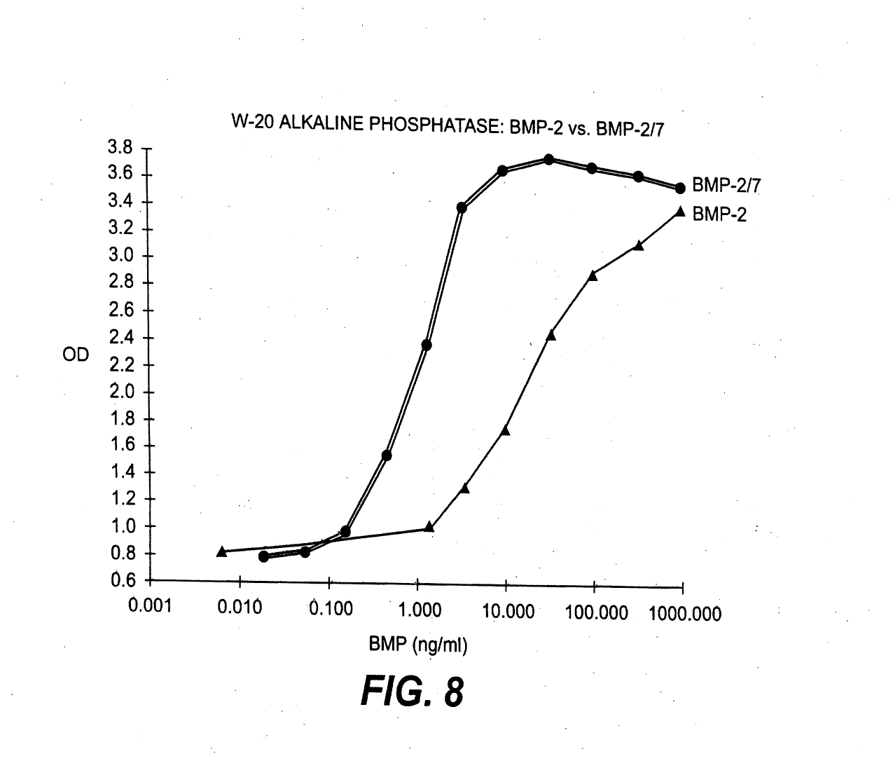

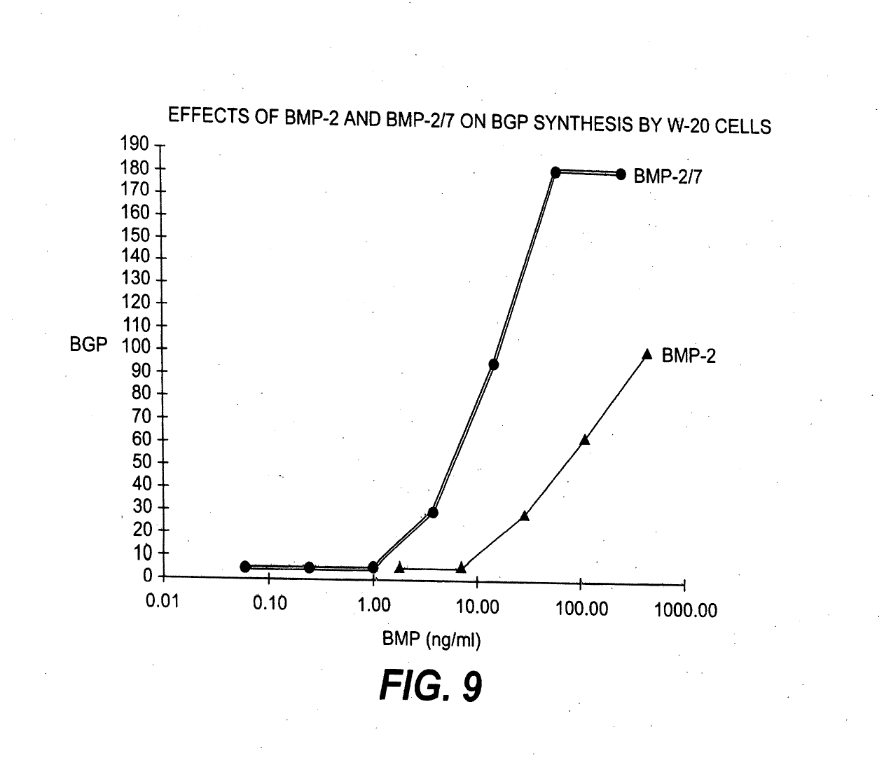

FIGS. 8 and 9 compare the activity of BMP-2 and BMP-2/7 in the W20 alkaline phosphatase and DGP (Bone Gla Protein, osteocalcin) assays. BMP-2/7 has greatly increased specific activity relative to BMP-2 (FIG. 8). From FIG. 8, approximately 1.3 ng/ml of BMP-2/7 was sufficient to induce 50% of the maximal alkaline phosphatase response in W-20 cells. A comparable value for BMP-2 is difficult to calculate, since the alkaline phosphatase response did not maximize, but greater than 30 ng/ml is needed for a half-maximal response. BMP-2/7 thus has a 20 to 30-fold higher specific activity than BMP-2 in the W-20 assay.

As seen in FIG. 9, BMP-2/7 was also a more effective stimulator of BGP (bone gla protein, osteocalcin) production-than BMP-2 in this experiment. Treating W-20-17 cells with BMP-2/7 for four days resulted in a maximal BGP response with 62 ng/ml, and 11 ng/ml elicits 50% of the maximal BGP response. In contrast, maximal stimulation of BGP synthesis by BMP-2. was not seen with doses up to 468 ng/ml of protein. The minimal dose of BMP-2/7 needed to elicit a BGP response by W-20-17 cells was 3.9 ng/ml, about seven-fold less than the 29 ng/ml required of BMP-2. These results were consistent with the data obtained in the W-20-17 alkaline phosphatase assays for BMP-2 and BMP-2/7.

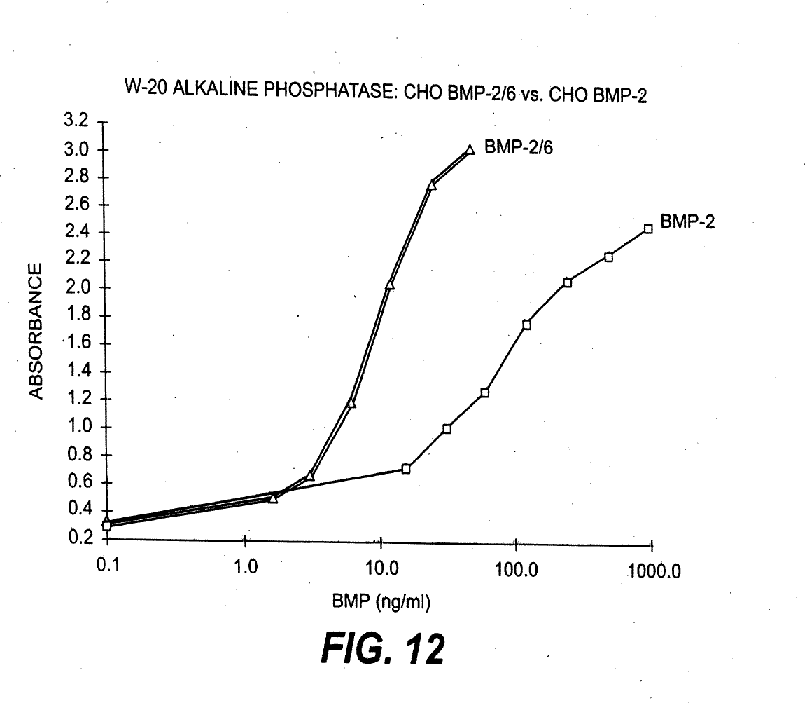

Preliminary analysis indicates that BMP-2/6 has a specific activity in vitro similar to that of BMP-2/7. The potencies of BMP-2 and BMP-2/6 on induction of alkaline phosphatase production in W-20 is compared, as shown in FIG. 12, BMP-2/6 has a higher specific activity than BMP-2 in this assay system. This data is in good agreement with data obtained from the in vivo assay of BMP-2 and BMP-2/6).

B. In Vivo Assay

(i) BMP-2/7

The purified BMP-2/7 and BMP-2 were tested in the rat ectopic bone formation assay. A series of different amounts of BMP-2/7 or BMP-2 were implanted in triplicate in rats. After 5 and 10 days, the implants were removed and examined histologically for the presence of bone and cartilage. The histological scores for the amounts of new cartilage and bone formed are summarized in Table A.

TABLE A

| | | 5 Day Implants | | | | | | 10 Day Implants | | | | | |
|---|---|---|---|---|---|---|---|---|---|---|---|---|---|
| | | BMP-2/7 | | | BMP-2 | | | BMP-2/7 | | | BMP-2 | | |
| 0.04 | C | ± | − | ± | − | − | − | ± | − | ± | − | − | − |
| | B | − | − | − | − | − | − | ± | − | ± | − | − | − |
| 0.02 | C | ± | 1 | ± | − | − | − | 2 | 1 | 2 | − | ± | ± |
| | B | − | − | − | − | − | − | 1 | ± | 1 | − | ± | − |
| 1.0 | C | 1 | ± | ± | ± | ± | ± | 2 | 2 | 2 | 1 | 1 | ± |
| | B | − | − | − | − | − | − | 2 | 3 | 3 | 1 | 1 | ± |
| 5.0 | C | 2 | 2 | 1 | 1 | ± | 1 | 1 | 1 | 2 | 1 | 2 | 1 |
| | B | ± | − | 1 | − | − | − | 4 | 4 | 3 | 2 | 3 | 2 |
| 25.0 | C | | | | | | | ± | ± | 2 | 2 | 2 | 2 |
| | B | | | | | | | 4 | 4 | 3 | 3 | 3 | 3 |

The amount of BMP-2/7 required to induce cartilage and bone in the rat ectopic assay is lower than that of BMP-2. Histologically, the appearance of cartilage and bone induced by BMP-2/7 and BMP-2 are identical.

(ii) BMP-2/6

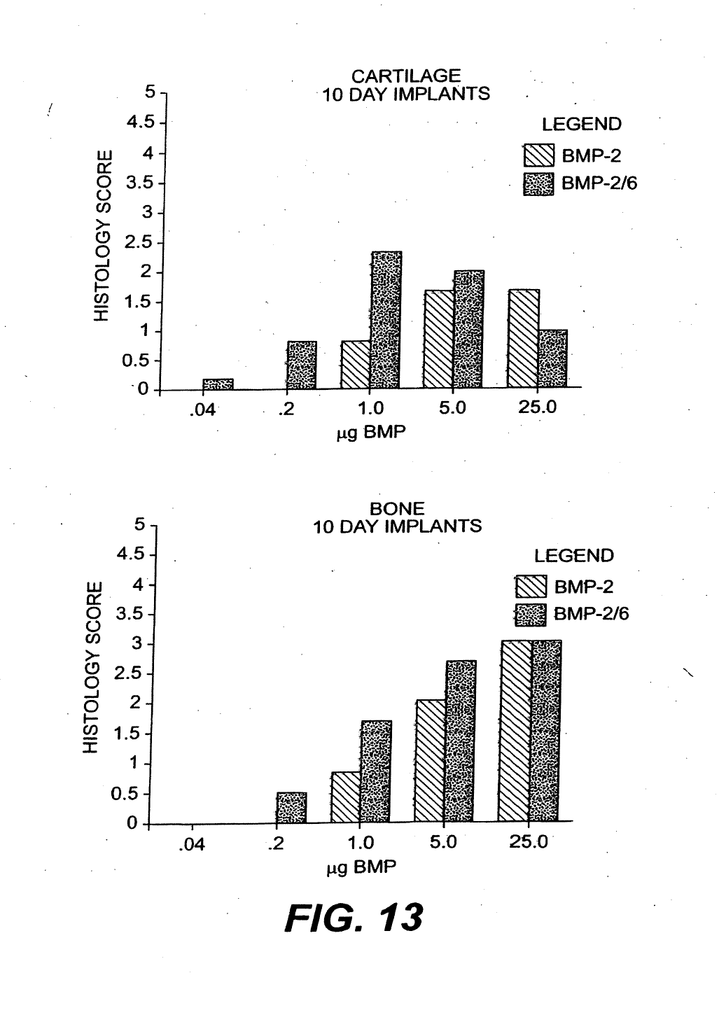

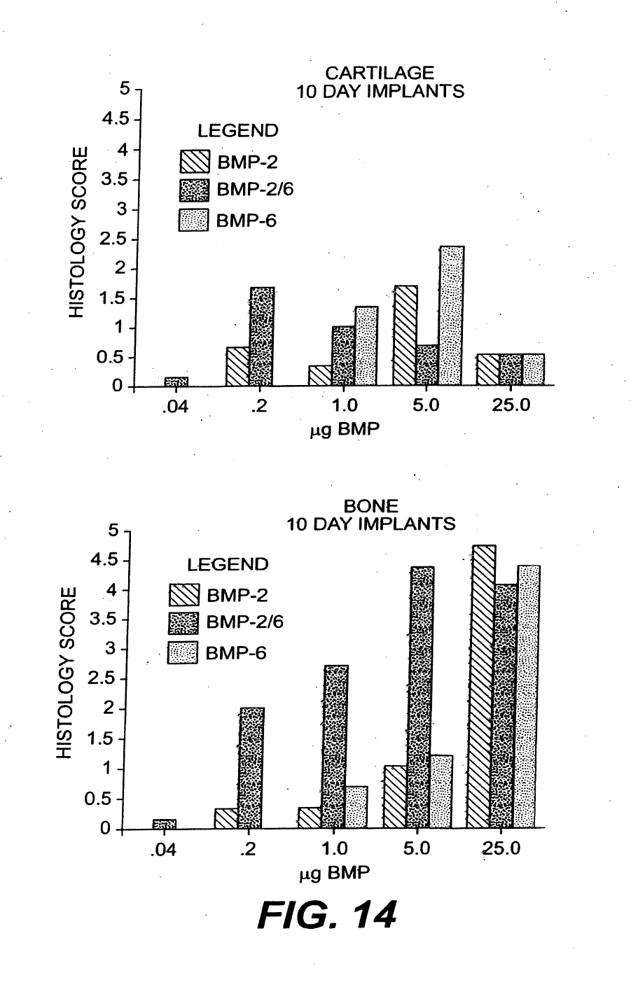

The in vivo activity of BMP-2/6 was compared with that of BMP-2 by implantation of various amounts of each BMP for ten days in the rat ectopic bone formation assay. The results of this study (Table B, FIG. 13) indicate that BMP-2/6, similar to BMP-2/7, has increased in vivo activity relative to BMP-2. The specific activities of BMP-2, BMP-6, and BMP-2/6 are compared in the ectopic bone formation assay ten days after the proteins are implanted. The results of these experiments are shown in Table C and FIG. 14. BMP-2/6 is a more potent inducer of bone formation than either BMP-2 or BMP-6. The amount of bone formation observed with BMP-2/6 was comparable to that observed with equivalent doses of BMP-2/7. The appearance of BMP-2/6 implants is quite similar to implants containing BMP-2 or BMP-2/7.

TABLE B
Histological scores of Implants of BMP 2/6 and
BMP-2 In rat ectopic assay (1O day implants).

| BMP (μg) | C/B | BMP-2/6 | | | BMP-2 | | |
|---|---|---|---|---|---|---|---|
| 0.04 | C | − | ± | − | − | − | − |
| | B | − | − | − | − | − | − |
| 0.20 | C | 1 | 1 | ± | − | − | − |
| | B | ± | ± | ± | − | − | − |
| 1.0 | C | 1 | 3 | 3 | 1 | 1 | ± |
| | B | 1 | 2 | 2 | 1 | 1 | ± |
| 5.0 | C | 2 | 2 | 2 | 1 | 2 | 2 |
| | B | 2 | 3 | 3 | 2 | 2 | 2 |
| 25. | C | 1 | 1 | 1 | 2 | 2 | 1 |
| | B | 3 | 3 | 3 | 3 | 3 | 3 |

TABLE C
Histological scores of implants of BMP-2, BMP-6, and
BMP-2/6 in rat ectopic assay (10 day implants).

| BMP (μg) | C/B | BMP-2 | | | BMP-6 | | | BMP-2/6 | | |
|---|---|---|---|---|---|---|---|---|---|---|
| 0.04 | C | − | − | − | − | − | − | − | − | ± |
| | B | − | − | − | − | − | − | − | − | ± |
| 0.20 | C | − | − | 2 | − | − | − | 1 | 2 | 2 |
| | B | − | − | 1 | − | − | − | 2 | 2 | 2 |
| 1.0 | C | − | ± | ± | 2 | 1 | 1 | 1 | 1 | 1 |
| | B | − | ± | ± | 1 | ± | ± | 3 | 3 | 2 |
| 5.0 | C | 2 | 2 | 1 | 3 | 1 | 3 | ± | ± | 1 |
| | B | 1 | 1 | 1 | 2 | ± | 1 | 4 | 5 | 4 |
| 25. | C | ± | ± | ± | ± | ± | ± | ± | ± | ± |
| | B | 5 | 4 | 5 | 4 | 4 | 5 | 4 | 5 | 3 |

### EXAMPLE 7

### Expression of BMP Dimer in E. Coli

A biologically active, homodimeric BMP-2 was expressed in E. coli using the techniques described in European Patent Application 433,255 with minor modifications. Other methods disclosed in the above-referenced European patent application may also be employed to produce heterodimers of the present invention from E. coli . Application of these methods to the heterodimers of this invention is anticipated to produce active BMP heterodimeric proteins from E. coli.

A. BMP-2 Expression Vector

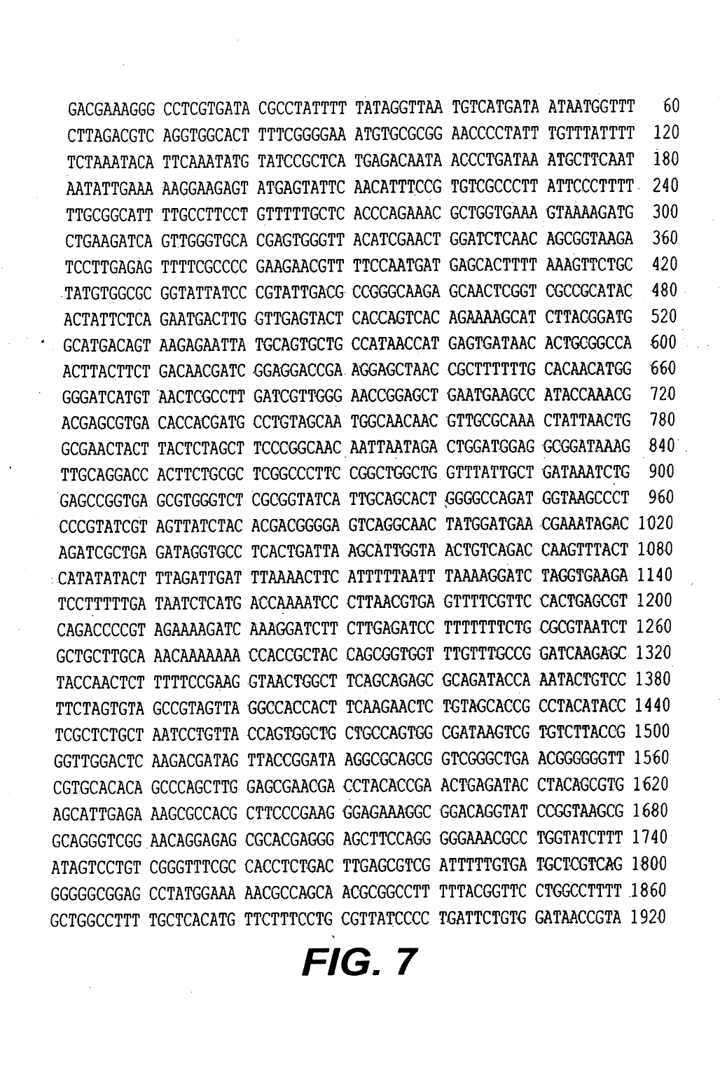

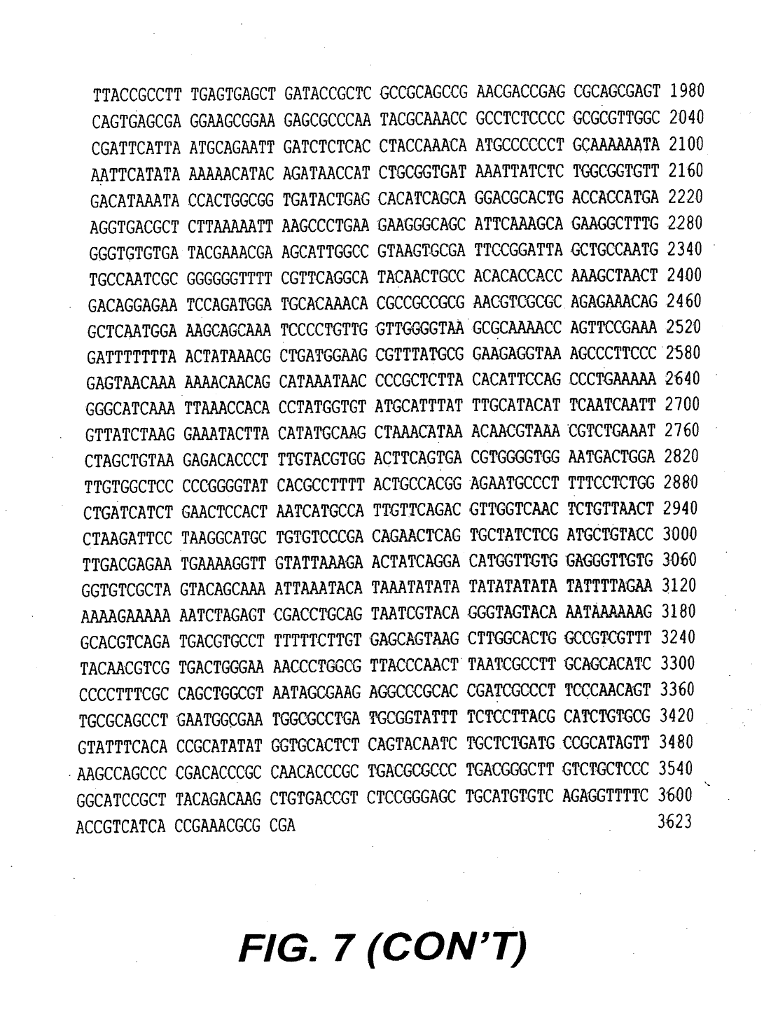

An expression plasmid pALBP2-781 (FIG. 7) (SEQ ID NO: 13) was constructed containing the mature portion of the BMP-2 (SEQ ID NO: 14) gene and other sequences which are described in detail below. This plasmid directed the accumulation of 5-10% of the total cell protein as BMP-2 in an E. coli host strain, GI724, described below.

Plasmid pALBP2-781 contains the following principal features. Nucleotides 1-2060 contain DNA sequences originating from the plasmid pUC-18 [Norrander et al, Gene, 26:101-106 (1983)] including-sequences containing the gene for β-lactamase which confers resistance to the antibiotic ampicillin in host E. coli strains, and a colE1-derived origin of replication. Nucleotides 2061-2221 contain DNA sequences for the major leftward promoter (pL) of bacteriophage λ [Sanger et al, J. Mol. Biol., 162:729-773 (1982)], including three operator sequences, O_{L}1, O_{L}2 and O_{L}3. The operators are the binding sites for λcI repressor protein, intracellular levels of which control the amount of transcription initiation from pL. Nucleotides 2222-2723 contain a strong ribosome binding sequence included on a sequence derived from nucleotides 35566 to 35472 and 38137 to 38361 from bacteriophage lambda as described in Sanger et. al., J. Mol. Biol., 162:729-773 (1982). Nucleotides 2724-3133 contain a DNA sequence encoding mature BMP-2 protein with an additional 62 nucleotides of 3′-untranslated sequence.

Nucleotides 3134-3149 provide a “Linker” DNA sequence containing restriction endonuclease sites. Nucleotides 3150-3218 provide a transcription termination sequence based on that of the E. coli aspA gene [Takagi et al, Nucl. Acids Res., 13:2063-2074 (1985)]. Nucleotides 3219-3623 are DNA sequences derived from pUC-18.

As described below, when cultured under the appropriate conditions in a suitable E. coli host strain, pALBP2-781 can direct the production of high levels (approximately 10% of the total cellular protein) of BMP-2 protein.

pALBP2-781 was transformed into the E. coli host strain GI724 (F, lacI^{q}, lacP^{L8}, ampc::λcI⁺) by the procedure of Dagert and Ehrlich, Gene, 6:23 (1979). [The untransformed host strain E. coli GI724 was deposited with the American Type Culture Collection, 12301 Parklawn Drive, Rockville, Md. on Jan. 31, 1991 under ATCC No. 55151 for patent purposes pursuant to applicable laws and regulations.] Transformants were selected on 1.5% w/v agar plates containing IMC medium, which is composed of M9 medium [Miller, “Experiments in Molecular Genetics”, Cold Spring Harbor Laboratory, New York (1972)] supplemented with 0.5% w/v glucose, 0.2% w/v casamino acids and 100 μg/ml ampicillin.

GI724 contains a copy of the wild-type λcI repressor gene stably integrated into the chromosome at the ampC locus, where it has been placed under the transcriptional control of Salmonella typhimurium trp promoter/operator sequences. In GI724, λcI protein is made only during growth in tryptophan-free media, such as minimal media or a minimal medium supplemented with casamino acids such as IMC, described above. Addition of tryptophan to a culture of GI724 will repress the trp promoter and turn off synthesis of λcI, gradually causing the induction of transcription from pL promoters if they are present in the cell.

G1724 transformed with pALBP2-781 was grown at 37° C. to an A₅₅₀ of 0.5 (Absorbence at 550 nm) in IMC medium. Tryptophan was added to a final concentration of 100 μg/ml and the culture incubated for a further 4 hours. During this time BMP-2 protein accumulated to approximately 10% of the total cell protein, all in the “inclusion body” fraction.

BMP-2 is recovered in a non-soluble, monomeric form as follows. Cell disruption and recovery is performed at 4° C. Approximately 9 g of the wet fermented E. coli GI724/pALBP2-781 cells are suspended in 30 mL of 0.1 M Tris/HCl, 10 mM EDTA, 1 mM phenyl methyl sulphonyl fluoride (PMSF), pH 8.3 (disruption buffer). The cells are passed four times through a cell disrupter and the volume is brought to 100 mL with the disruption buffer. The suspension is centrifuged for 20 min. (15,000×g). The pellet obtained is suspended in 50 mL disruption buffer containing 1 M NaCl and centrifuged for 10 min. as above. The pellet is suspended in 50 mL disruption buffer containing 1% Triton X-100 (Pierce) and again centrifuged for 10 min. as above. The washed pellet is then suspended in 25 mL of 20 mM Tris/HCl, 1 mM EDTA, 1 mM PMSF, 1% DTT, pH 8.3 and homogenized in a glass homogenizer. The resulting suspension contains crude monomeric BMP-2 in a non-soluble form.

Ten mL of the. BMP-2 suspension, obtained as described above, are acidified with 10% acetic acid to pH 2.5 and centrifuged in an Eppendorf centrifuge for 10 min. at room temperature. The supernatant is chromatographed. Chromatography was performed on a Sephacryl S-100 HR column (Pharmacia, 2.6×83 cm) in 1% acetic acid at a flow rate of 1.4 mL/minute. Fractions containing monomeric, BMP-2 are pooled. This material is used to generate biologically active, homodimer BMP-2.

Biologically active, homodimeric BMP-2 can be generated from the monomeric BMP-2 obtained following solubilization and purification, described above, as follows.

0.1, 0.5 or 2.5 mg of the BMP-2 is dissolved at a concentration of 20, 100 or 500 μg/mL, respectively, in 50 mM Tris/HCl, pH 8.0, 1 M NaCl, 5 mM EDTA, 2 mM reduced glutathione, 1 mM oxidized glutathione and 33 mM CHAPS [Calbiochem]. After 4 days at 4° C. or 23° C., the mixture is diluted 5 to 10 fold with 0.1% TFA.

Purification of biologically active BMP-2 is achieved by subjecting the diluted mixture to reverse phase HPLC on a a Vydac C4 214TP54 column (25×0.46 cm) [The NEST Group, USA] at a flow rate of 1 ml/minute. Buffer A is 0.1% TFA. Buffer B is 90% acetonitrile, and 0.1% TFA. The linear gradient was 0 to 5 minutes at 20% Buffer B; 5 to 10 minutes at 20 to 30 % Buffer B; 10 to 40 minutes at 30 to 60% Buffer B; and 40 to 50 minutes at 60 to 100% Buffer B. Homodimeric BMP-2 is eluted and collected from the HPLC column.

The HPLC fractions are lyophilized to dryness, redissolved in sample buffer (1.5 M Tris-HCl, pH 8.45, 12% glycerol, 4% SDS, 0.0075% Serva Blue G, 0.0025% Phenol Red, with or without 100 mM dithiothreitol) and heated for five minutes at 95° C. The running buffer is 100 mM Tris, 100 mM tricine (16% tricine gel) [Novex], 0.1% SDS at pH 8.3. The SDS-PAGE gel is run at 125 volts for 2.5 hours.

The gel is stained for one hour with 200 ml of 0.5% Coomassie Brilliant Blue R-250, 25% isopropanol, 10% acetic acid, heated to 60° C. The gel is then destained with 10% acetic acid, 10% isopropanol until the background is clear.

The reduced material ran at approximately 13 kD; the non-reduced material ran at approximately 30 kD, which is indicative of the BMP-2 diner. This material was later active in the W20 assay of Example 8.

B. BMP-7 Expression Vector

For high level expression of BMP-7 a plasmid pALBMP7-981 was constructed. pAlBMP7-981 is identical to plasmid pALBP2-781 with two exceptions: the BMP-2 gene (residues 2724-3133 of pALBP2-781) is replaced by the mature portion of the BMP-7 gene, deleted for sequenced encoding the first seven residues of the mature BMP-7 protein sequence:

ATGTCTCATAATC GTTCTAAAAC TCCAAAAAAT CAAGAAGCTC
TGCGTATGGC CAACGTGGCA GAGAACAGCA GCAGCGACCA
GAGGCAGGCC TGTAAGAAGC ACGAGCTGTA TGTCAGCTTC
CGAGACCTGG GCTGGCAGGA CTGGATCATC GCGCCTGAAG
GCTACGCCGC CTACTACTGT GAGGGGGAGT GTGCCTTCCC
TCTGAACTCC TACATGAACG CCACCAACCA CGCCATCGTG
CAGACGCTGG TCCACTTCAT CAACCCGGAA ACGGTGCCCA
AGCCCTGCTG TGCGCCCACG CAGCTCAATG CCATCTCCGT
CCTCTACTTC GATGACAGCT CCAACGTCAT CCTGAAGAAA
TACAGAAACA TGGTGGTCCG GGCCTGTGGC TGCCACTAGC
TCCTCCGAGA ATTCAGACCC TTTGGGGCCA AGTTTTTCTG
GATCCT

and the ribosome binding site found between residues 2707 and 2723 in pALBP2-781 is replaced by a different ribosome binding site, based on that found preceding the T7 phage gene 10, of sequence 5′-CAAGAAGGAGATATACAT-3′. The host strain and growth conditions used for the production of BMP-7 were as described for BMP-2.
C. BMP-3 Expression Vector

For high level expression of BMP-3 a plasmid pALB3-782 was constructed. This plasmid is identical to plasmid pALBP2-781, except that the BMP-2 gene (residues 2724-3133 of pALBP2-781) is replaced by a gene encoding a form of mature BMP-3. The sequence of this BMP-3 gene is:

ATGCGTAAAC AATGGATTGA ACCACGTAAC TGTGCTCGTC
GTTATCTGAA AGTAGACTTT GCAGATATTG GCTGGAGTGA
ATGGATTATC TCCCCCAAGT CCTTTGATGC CTATTATTGC
TCTGGAGCAT GCCAGTTCCC CATGCCAAAG TCTTTGAAGC
CATCAAATCA TGCTACCATC CAGAGTATAG TGAGAGCTGT
GGGGGTCGTT CCTGGGATTC CTGAGCCTTG CTGTGTACCA
GAAAAGATGT CCTCACTCAG TATTTTATTC TTTGATGAAA
ATAAGAATGT AGTGCTTAAA GTATACCCTA ACATGACAGT
AGAGTCTTGC GCTTGCAGAT AACCTGGCAA AGAACTCATT
TGAATGCTTA ATTCAAT

The host strain and growth conditions used for the production of BMP-3 were as described for BMP-2.

D. Expression of a BMP-2/7 Heterodimer in E. coli

Denatured and purified E. coli BMP-2 and BMP-7 monomers were isolated from E. coli inclusion body pellets by acidification and gel filtration as previously as previously described above. 125 ug of each BMP in 1% acetic acid were mixed and taken to dryness in a speed vac. The material was resuspended in 2.5 ml 50 mM Tris, 1.0 NaCl, 5 mM EDTA, 33 mM CHAPS, 2 mM glutathione (reduced), 1 mM glutathione (oxidized), pH 8.0. The sample was incubated at 23 C for one week.

The BMP-2/7 heterodimer was isolated by HPLC on a 25×0.46 cm Vydac C4 column. The sample was centrifuged in a microfuqe for 5 minutes, and the supernatant was diluted with 22.5 ml 0.1% TFA.

| | |
|---|---|
| A buffer | 0.1% TFA |
| B buffer | 0.1% TFA, 95% acetonitrile |

1.0 ml/minute

| | |
|---|---|
| 0-5′ | 20% B |
| 5-10′ | 20-30% B |
| 10-90′ | 30-50% B |
| 9O-100′ | 50-100% B |

By SDS-PAGE analysis, the BMP-2/7 heterodimer eluted at about 23′.

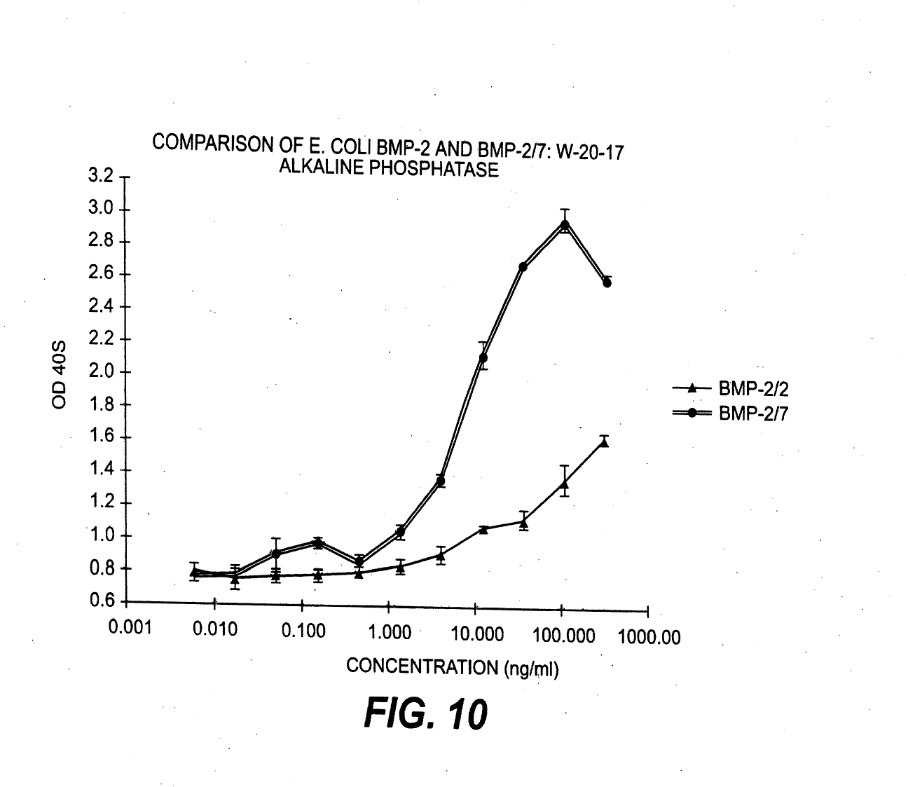

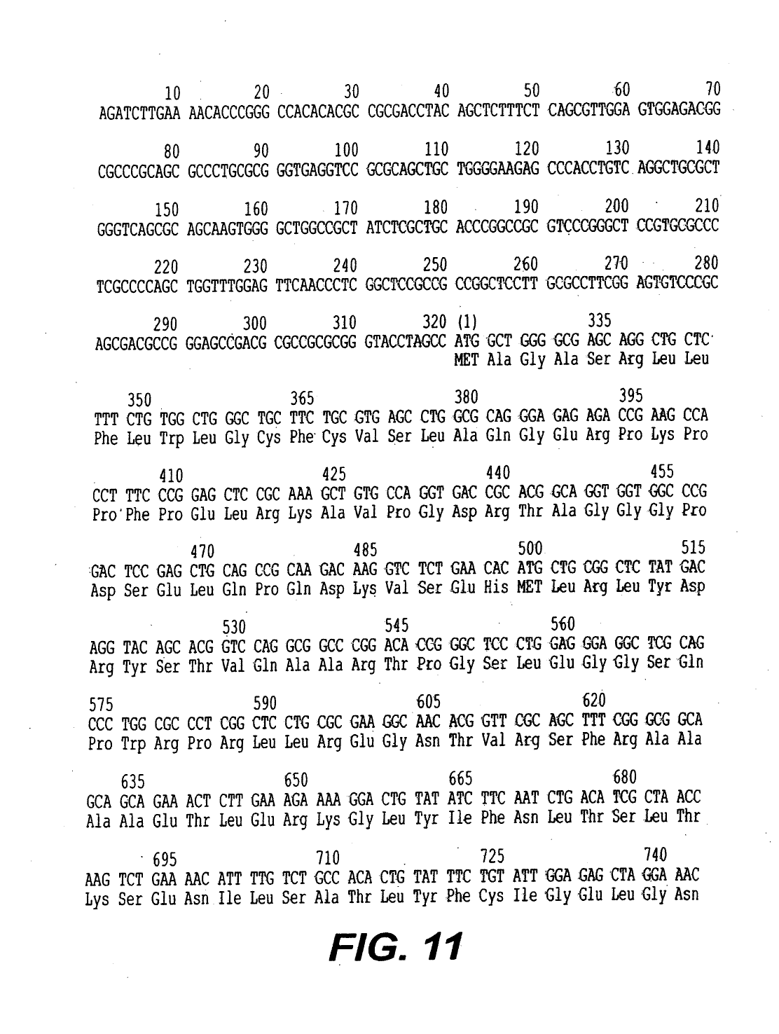

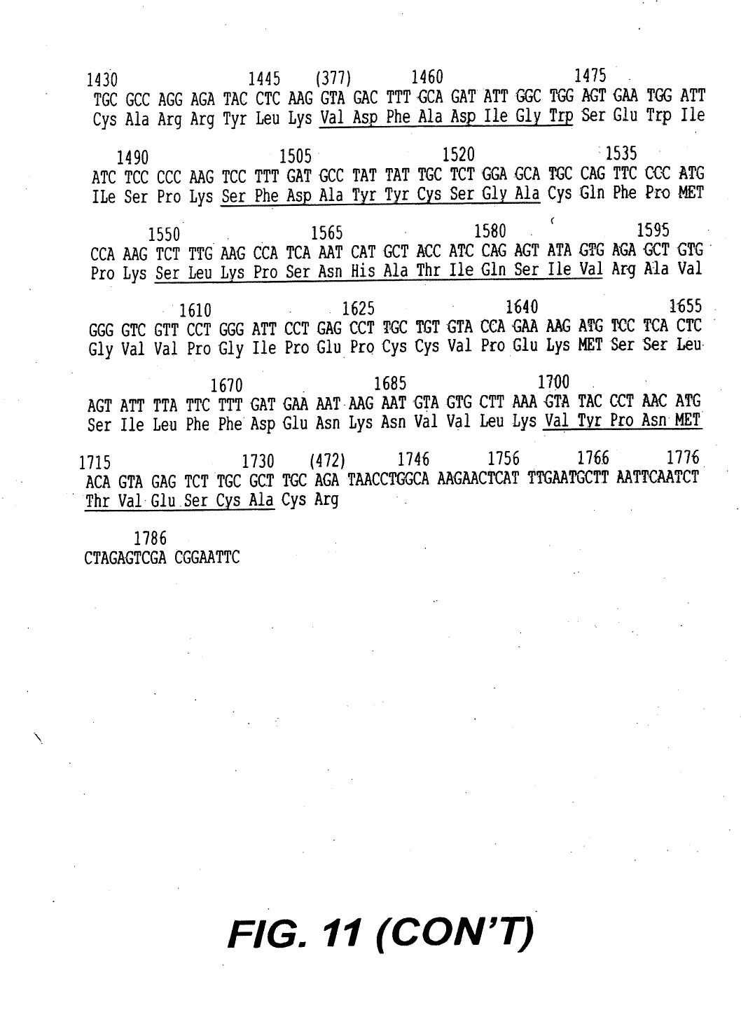

FIG. 10 is a comparison of the W-20 activity of E. coli BMP-2 and BMP-2/7 heterodimer, indicating greater activity of the heterodimer.

F. Expression of BMP-2/3 Heterodimer in E. coli

BMP-2 and BMP-3 monomers were isolated as follows: to 1.0 g of frozen harvested cells expressing either BMP-2 or BMP-3 was added 3.3 ml of 100 mM Tris, 10 mM EDTA, pH 8.3. The cells were resuspended by vortexing vigorously. 33 ul of 100 mM PMSF in isopropanol was added and the cells lysed by one pass through a French pressure cell. The lysate was centrifuged in a microfuge for 20 minutes at 4 C. The supernatant was discarded. The inclusion body pellet was taken up in 8.0 M quanidine hydrochloride, 0.25 M OTT, 0.5 M Tris, 5 mM EDTA, pH 8.5, and heated at 37 C for one hour.

The reduced and denatured BMP monomers were isolated by HPLC on a Supelco C4 guard column as follows:

| | |
|---|---|
| A buffer | 0.1% TFA |
| B buffer | 0.1% TFA, 95% acetonitrile |

1.0 ml/minute

| | |
|---|---|
| 0-5′ | 1% B |
| 5-40′ | 1-70% B |
| 40-45′ | 70-100% B |

Monomeric BMP eluted at 28-30′. Protein concentration was estimated by A280 and the appropriate extinction coefficient.

10 ug of BMP-2 and BMP-3 were combined and taken to dryness in a speed vac. To this was added 50 ul of 50 mM Tris, 1.0 M NaCl, 5 mM EDTA, 33 mM CHAPS, 2 mM reduced glutathione, 1 mM oxidized glutathione, pH 8.5. The sample was incubated at 23 for 3 days. The sample was analyzed by SDS-PAGE on a 16% tricine gel under reducing and nonreducing conditions. The BMP-2/3 heterodimer migrated at about 35 kd nonreduced, and reduced to BMP-2 monomer at about 13 kd and BMP-3 monomer at about 21 kd.

BMP-2/3 heterodimer produced in E. coli is tested for in vivo activity. (20 μg) at (ten days) is utilized to compare the in vivo activity of BMP-2/3 to BMP-2. BMP-2/3 implants showed no cartilage or bone forming activity, while the BMP-2 control implants showed the predicted amounts of bone and cartilage formation. The in vivo data obtained with BMP-2/3 is consistent with the in vitro data from the W-20 assay.

### EXAMPLE 8

### W-20 Bioassays

A. Description of W-20 Cells

Use of the W-20 bone marrow stromal cells as an indicator cell line is based upon the conversion of these cells to osteoblast-like cells after treatment with BMP-2 [R. S. Thies et al, “Bone Morphogenetic Protein alters W-20 stromal cell differentiation in vitro”, Journal of Bone and Mineral Research, 5(2):305 (1990); and R. S. Thies et al, “Recombinant Human Bone Morphogenetic Protein 2 Induces Osteoblastic Differentiation in W-20-17 Stromal Cells”, Endocrinology, in press (1992)]. Specifically, W-20 cells are a clonal bone marrow stromal cell line derived from adult mice by researchers in the laboratory of Dr. D. Nathan, Children's Hospital, Boston, Mass. BMP-2 treatment of W-20 cells results in (1) increased alkaline phosphatase production, (2) induction of PTH stimulated cAMP, and (3) induction of osteocalcin synthesis by the cells. While (1) and (2) represent characteristics associated with the osteoblast phenotype, the ability to synthesize osteocalcin is a phenotypic property only displayed by mature osteoblasts. Furthermore, to date we have observed conversion of W-20 stromal cells to osteoblast-like cells only upon treatment with BMPs. In this manner, the in vitro activities displayed by BMP treated W-20 cells correlate with the in vivo bone forming activity known for BMPS.

Below two in vitro assays useful in comparison of BMP activities of novel osteoinductive molecules are described.

B. W-20 Alkaline Phosphatase Assay Protocol

W-20 cells are plated into 96 well tissue culture plates at a density of 10,000 cells per well in 200 μl of media (DME with 10% heat inactivated fetal calf serum, 2 mM glutamine and 100 U/ml+100 μg/ml streptomycin. The cells,are allowed to attach overnight in a 95% air, 5% CO₂ incubator at 37° C.

The 200 μl of media is removed from each well with a multichannel pipettor and replaced with an equal volume of test sample delivered in DME with 10% heat inactivated fetal calf serum, 2 mM glutamine and 1% penicillin-streptomycin. Test substances are assayed in triplicate.

The test samples and standards are allowed a 24 hour incubation period with the W-20 indicator cells. After the 24 hours, plates are removed from the 37° C. incubator and the test media are removed from the cells.

The W-20 cell layers are washed 3 times with 200 μl per well of calcium/magnesium free phosphate buffered saline and these washes are discarded.

50 μl of glass distilled water is added to each well and the assay plates are then placed on a dry ice/ethanol bath for quick freezing. Once frozen, the assay plates are removed from the dry ice/ethanol bath and thawed at 37° C. This step is repeated 2 more times for a total of 3 freeze-thaw procedures. Once complete, the membrane bound alkaline phosphatase is available for measurement.

50 μl of assay mix (50 mM glycine, 0.05% Triton X-100, 4 MM MgCl₂, 5 mM p-nitrophenol phosphate, pH=10.3) is added to each assay well and the assay plates are then incubated for 30 minutes at 37° C. in a shaking waterbath at 60 oscillations per minute.

At the end of the 30 minute incubation, the reaction is stopped by adding 100 μl of 0.2 N NaOH to each well and placing the assay plates on ice.

The spectrophotometric absorbance for each well is read at a wavelength of 405 nanometers. These values are then compared to known standards to give an estimate of the alkaline phosphatase activity in each sample. For example, using known amounts of p-nitrophenol phosphate, absorbance values are generated.

This is shown in Table I.

TABLE I
Absorbance Values for Known Standards
of P-Nitrophenol Phosphate

| P-nitrophenol phosphate umoles | Mean absorbance (405 nm) |
|---|---|
| 0.000 | 0 |
| 0.006 | 0.261 +/− .024 |
| 0.012 | 0.521 +/− .031 |
| 0.018 | 0.797 +/− .063 |
| 0.024 | 1.074 +/− .061 |
| 0.030 | 1.305 +/− .083 |

Absorbance values for known amounts of BMP-2 can be determined and converted to μmoles of p-nitrophenol phosphate cleaved per unit time as shown in Table II.

TABLE II
Alkaline Phosphatase Values for W-20 Cells Treating with BMP-2

| BMP-2 concentration | Absorbance Reading | umoles substrate |
|---|---|---|
| ng/ml | 4O5 nmeters | per hour |
| 0 | 0.645 | 0.024 |
| 1.56 | 0.696 | 0.026 |
| 3.12 | 0.765 | 0.029 |
| 6.25 | 0.923 | 0.036 |
| 12.50 | 1.121 | 0.044 |
| 25.0 | 1.457 | 0.058 |
| 50.0 | 1.662 | 0.067 |
| 100.0 | 1.977 | 0.080 |

These values are then used to compare the activities of known amounts of BMP heterodimers to BMP-2 homodimer.

C. Osteocalcin RIA Protocol

W-20 cells are plated at 10⁶ cells per well in 24 well multiwell tissue culture dishes in 2 mls of DME containing 10% heat inactivated fetal calf serum, 2 mM glutamine. The cells are allowed to attach overnight in an atmosphere of 95% air 5% CO₂ at 37° C.

The next day the medium is changed to DME containing 10% fetal calf serum, 2 mM glutamine and the test substance in a total volume of 2 ml. Each test substance is administered to triplicate wells. The test substances are incubated with the W-20 cells for a total of 96 hours with replacement at 48 hours by the same test medias.

At the end of 96 hours, 50 μl of the test media is removed from each well and assayed for osteocalcin production using a radioimmunoassay for mouse osteocalcin. The details of the assay are described in the kit manufactured by Biomedical Technologies Inc., 378 Page Street, Stoughton, Mass. 02072. Reagents for the assay are found as product numbers BT-431 (mouse osteocalcin standard), BT-432 (Goat anti-mouse Osteocalcin), BT-431R (iodinated mouse osteocalcin), BT-415 (normal goat serum) and BT-414 (donkey anti goat IgG). The RIA for osteocalcin synthesized by W-20 cells in response to BMP treatment is carried out as described in the protocol provided by the manufacturer.

The values obtained for the test samples are compared to values for known standards of mouse osteocalcin and to the amount of osteocalcin produced by W-20 cells in response to challenge with known amounts of BMP-2. The values for BMP-2 induced osteocalcin synthesis by W-20 cells is shown in Table III.

TABLE III
Osteocalcin Synthesis by W-2O Cells

| BMP-2 Concentration ng/ml | Osteocalcin Synthesis ng/well |
|---|---|
| 0 | 0.8 |
| 2 | 0.9 |
| 4 | 0.8 |
| 8 | 2.2 |
| 16 | 2.7 |
| 31 | 3.2 |
| 62 | 5.1 |
| 125 | 6.5 |
| 250 | 8.2 |
| 500 | 9.4 |
| 1000 | 10.0 |

### EXAMPLE 9

### Rosen Modified Sampath-Reddi Assay

A modified version of the rat bone formation assay described in Sampath and Reddi, Proc. Natl. Acad. Sci. USA, 80:6591-6595 (1983) is used to evaluate bone and/or cartilage activity of BMP proteins. This modified assay is herein called the Rosen-modified Sampath-Reddi assay. The ethanol precipitation step of the Sampath-Reddi procedure is replaced by dialyzing (if the composition is a solution) or diafiltering (if the composition is a suspension) the fraction to be assayed against water. The solution or suspension is then redissolved in 0.1% TFA, and the resulting solution added to 20 mg of rat matrix. A mock rat matrix sample not treated with the protein serves as a control. This material is frozen and lyophilized and the resulting powder enclosed in #5 gelatin capsules. The capsules are implanted subcutaneously in the abdominal thoracic area of 21-49 ay old male Long Evans rats. The implants are removed after 7-14 days. Half of each implant is used for alkaline phosphatase analysis [see, A. H. Reddi et al, Proc. Natl. Acad. Sci., 69:1601 (1972)].

The other half of each implant is fixed and processed for histological analysis. 1 μm glycolmethacrylate sections are stained with Von Kossa and acid fuschin to score the amount of induced bone and cartilage formation present in each implant. The terms +1 through +5 represent the area of each histological section of an implant occupied by new bone and/or cartilage cells and matrix. A score of +5 indicates that greater than 50% of the implant is new bone and/or cartilage produced as a direct result of protein in the implant. A score of +4, +3, +2, and +1 would indicate that greater than 40%, 30%, 20% and 10% respectively of the implant contains new cartilage and/or bone.

The heterodimeric BMP proteins of this invention may be assessed for activity on this assay.

Numerous modifications and variations in practice of this invention are expected to occur to those skilled in the art. Such modifications and variations are encompassed within the following claims.

1-36. (canceled)

37. An isolated recombinant heterodimeric protein having bone stimulating activity comprising a bone morphogenetic protein-2 comprising amino acids 283-396 of SEQ ID NO: 2, or a fragment thereof, associated by at least one disulfide bond with a bone morphogenetic protein-5 comprising amino acids 329-454 of SEQ ID NO: 10, or a fragment thereof.

38. A method of treating a bone or cartilage disorder in a patient comprising administering the heterodimeric protein of claim 37.


SEQUENCE LISTING (30 sequences)

SEQ ID NO: 1 - DNA (genomic), 1607 bp; double-stranded, unknown
  CDS 356..1543
gtcgactctagagtgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaacttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaa

SEQ ID NO: 2 - protein, 396 aa
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWETFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA (genomic), 1954 bp; double-stranded, unknown
  CDS 403..1626
ctctagagggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaatggaaaaaatccctaaacattcaccttgaccttatttatgactttacgtgcaaatgttttgaccatattgatcatatattttgacaaaatatatttataactacgtattaaaagaaaaaaataaaatgagtcattattttaaaaaaaaaaaaaaaactctagagtcgacggaattc

SEQ ID NO: 4 - protein, 408 aa
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 5 - DNA (genomic), 1448 bp; double-stranded, unknown
  CDS 97..1389
gtgaccgagcggcgcggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctc

SEQ ID NO: 6 - protein, 431 aa
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 7 - DNA (cDNA to mRNA), 2923 bp, Homo sapiens; double-stranded, circular
  CDS 160..1701
  mat_peptide 1282..1698
  mRNA 1..2923
  hypothetical: NO
  original source tissue type: Human placenta
  immediate source library: Stratagene catalog #936203 Human placenta cDNA library
  immediate source clone: BMP6C35
  units: bp
cgaccatgagagataaggactgagggccaggaaggggaagcgagcccgccgagaggtggcggggactgctcacgccaagggccacagcggccgcgctccggcctcgctccgccgctccacgcctcgcgggatccgcgggggcagcccggccgggcggggatgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgataagcagcccttcatggtggctttcttcaaagtgagtgaggtccacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaactcgaaaccagatgctggggacacacattctgccttggattcctagattacatctgccttaaaaaaacacggaagcacagttggaggtgggacgatgagactttgaaactatctcatgccagtgccttattacccaggaagattttaaaggacctcattaataatttgctcacttggtaaatgacgtgagtagttgttggtctgtagcaagctgagtttggatgtctgtagcataaggtctggtaactgcagaaacataaccgtgaagctcttcctaccctcctcccccaaaaacccaccaaaattagttttagctgtagatcaagctatttggggtgtttgttagtaaatagggaaaataatctcaaaggagttaaatgtattcttggctaaaggatcagctggttcagtactgtctatcaaaggtagattttacagagaacagaaatcggggaagtggggggaacgcctctgttcagttcattcccagaagtccacaggacgcacagcccaggccacagccagggctccacggggcgcccttgtctcagtcattgctgttgtatgttcgtgctggagttttgttggtgtgaaaatacacttatttcagccaaaacataccatttctacacctcaatcctccatttgctgtactctttgctagtaccaaaagtagactgattacactgaggtgaggctacaaggggtgtgtaaccgtgtaacacgtgaaggcagtgctcacctcttctttaccagaacggttctttgaccagcacattaacttctggactgccggctctagtaccttttcagtaaagtggttctctgcctttttactatacagcataccacgccacagggttagaaccaacgaagaaaataaaatgagggtgcccagcttataagaatggtgttagggggatgagcatgctgtttatgaacggaaatcatgatttccctgtagaaagtgaggctcagattaaattttagaatattttctaaatgtctttttcacaatcatgtgactgggaaggcaatttcatactaaactgattaaataatacatttataatctacaactgtttgcacttacagctttttttgtaaatataaactataatttattgtctattttatatctgttttgctgtggcgttggggggggggccgggcttttgggggggggggtttgtttggggggtgtcgtggtgtgggcgggcgg

SEQ ID NO: 8 - protein, 513 aa
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 9 - DNA, 2153 bp, Homo sapiens; double-stranded, linear
  CDS 699..2063
  mat_peptide 1647..2060
  mRNA 1..2153
  hypothetical: NO
  original source cell line: U2-OS osteosarcoma
  immediate source library: U2-OS human osteosarcoma cDNA library
  immediate source clone: U2-16
  units: bp
ctggtatatttgtgcctgctggaggtggaattaacagtaagaaggagaaagggattgaatggacttacaggaaggatttcaagtaaattcagggaaacacatttacttgaatagtacaacctagagtattattttacactaagacgacacaaaagatgttaaagttatcaccaagctgccggacagatatatattccaacaccaaggtgcagatcagcatagatctgtgattcagaaatcaggatttgttttggaaagagctcaagggttgagaagaactcaaaagcaagtgaagattactttgggaactacagtttatcagaagatcaacttttgctaattcaaataccaaaggcctgattatcataaattcatataggaatgcataggtcatctgatcaaataatattagccgtcttctgctacatcaatgcagcaaaaactcttaacaactgtggataattggaaatctgagtttcagctttcttagaaataactactcttgacatattccaaaatatttaaaataggacaggaaaatcggtgaggatgttgtgctcagaaatgtcactgtcatgaaaaataggtaaatttgttttttcagctactgggaaactgtacctcctagaaccttaggtttttttttttttaagaggacaagaaggactaaaaatatcaacttttgcttttggacaaaaatgcatctgactgtatttttacttaagggtattgtgggtttcctctggagctgctgggttctagtgggttatgcaaaaggaggtttgggagacaatcatgttcactccagttttatttatagaagactacggaaccacgaaagacgggaaatacaaagggaaattctctctatcttgggtttgcctcacagacccagaccattttcacctggaaaaatgaccaatcaagcgtcctctgcacctctctttatgctggatctctacaatgccgaagaaaatcctgaagagtcggagtactcagtaagggcatccttggcagaagagaccagaggggcaagaaagggatacccagcctctcccaatgggtatcctcgtcgcatacagttatctcggacgactcctctgaccacccagagtcctcctctagccagcctccatgataccaactttctgaatgatgctgacatggtcatgagctttgtcaacttagttgaaagagacaaggatttttctcaccagcgaaggcattacaaagaatttcgatttgatcttacccaaattcctcatggagaggcagtgacagcagctgaattccggatatacaaggaccggagcaacaaccgatttgaaaatgaaacaattaagattagcatatatcaaatcatcaaggaatacacaaatagggatgcagatctgttcttgttagacacaagaaaggcccaagctttagatgtgggttggcttgtctttgatatcactgtgaccagcaatcattgggtgattaatccccagaataatttgggcttacagctctgtgcagaaacaggggatggacgcagtatcaacgtaaaatctgctggtcttgtgggaagacagggacctcagtcaaaacaaccattcatggtggccttcttcaaggcgagtgaggtacttcttcgatccgtgagagcagccaacaaacgaaaaaatcaaaaccgcaataaatccagctctcatcaggactcctccagaatgtccagtgttggagattataacacaagtgagcaaaaacaagcctgtaagaagcacgaactctatgtgagcttccgggatctgggatggcaggactggattatagcaccagaaggatacgctgcattttattgtgatggagaatgttcttttccacttaacgcccatatgaatgccaccaaccacgctatagttcagactctggttcatctgatgtttcctgaccacgtaccaaagccttgttgtgctccaaccaaattaaatgccatctctgttctgtactttgatgacagctccaatgtcattttgaaaaaatatagaaatatggtagtacgctcatgtggctgccactaatattaaataatattgataataacaaaaagatctgtattaaggtttatggctgcaataaaaagcatactttcagacaaacagaaaaaaaaa

SEQ ID NO: 10 - protein, 454 aa
MHLTVFLLKGIVGFLWSCWVLVGYAKGGLGDNHVHSSFIYRRLRNHERREIQREILSILGLPHRPRPFSPGKMTNQASSAPLFMLDLYNAEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 11 - DNA (cDNA to mRNA), 1003 bp, Homo sapiens; double-stranded, circular
  CDS 8..850
  mat_peptide 427..843
  mRNA 1..997
  hypothetical: NO
  original source tissue type: Human Heart
  immediate source library: Human heart cDNA library stratagene catalog #936208
  immediate source clone: hH38
  units: bp
gaattccgagccccattggaaggagttccgctttgacctgacccagatcccggctggggaggcggtcacagctgcggagttccggatttacaaggtgcccagcatccacctgctcaacaggaccctccacgtcagcatgttccaggtggtccaggagcagtccaacagggagtctgacttgttctttttggatcttcagacgctccgagctggagacgagggctggctggtgctggatgtcacagcagccagtgactgctggttgctgaagcgtcacaaggacctgggactccgcctctatgtggagactgaggatgggcacagcgtggatcctggcctggccggcctgctgggtcaacgggccccacgctcccaacagcctttcgtggtcactttcttcagggccagtccgagtcccatccgcacccctcgggcagtgaggccactgaggaggaggcagccgaagaaaagcaacgagctgccgcaggccaaccgactcccagggatctttgatgacgtccacggctcccacggccggcaggtctgccgtcggcacgagctctacgtcagcttccaggaccttggctggctggactgggtcatcgccccccaaggctactcagcctattactgtgagggggagtgctccttcccgctggactcctgcatgaacgccaccaaccacgccatcctgcagtccctggtgcacctgatgaagccaaacgcagtccccaaggcgtgctgtgcacccaccaagctgagcgccacctctgtgctctactatgacagcagcaacaacgtcatcctgcgcaagcaccgcaacatggtggtcaaggcctgcggctgccactgagtcagcccgcccagccctactgcagccacccttctcatctggatcgggccctgcagaggcagaaaacccttaaatgctgtcacagctcaagcaggagtgtcaggggccctcactctcggtgcctacttcctgtcaggcttctgggaattc

SEQ ID NO: 12 - protein, 281 aa
EPHWKEFRFDLTQIPAGEAVTAAEFRIYKVPSIHLLNRTLHVSMFQVVQEQSNRESDLFFLDLQTLRAGDEGWLVLDVTAASDCWLLKRHKDLGLRLYVETEDGHSVDPGLAGLLGQRAPRSQQPFVVTFFRASPSPIRTPRAVRPLRRRQPKKSNELPQANRLPGIFDDVHGSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 13 - DNA (genomic), 3623 bp; double-stranded, linear
  CDS 2724..3071
  terminator 3150..3218
  RBS 2222..2723
  immediate source clone: pALBP2-781
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagaattgatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcagcattcaaagcagaaggctttggggtgtgtgatacgaaacgaagcattggccgtaagtgcgattccggattagctgccaatgtgccaatcgcggggggttttcgttcaggactacaactgccacacaccaccaaagctaactgacaggagaatccagatggatgcacaaacacgccgccgcgaacgtcgcgcagagaaacaggctcaatggaaagcagcaaatcccctgttggttggggtaagcgcaaaaccagttccgaaagatttttttaactataaacgctgatggaagcgtttatgcggaagaggtaaagcccttcccgagtaacaaaaaaacaacagcataaataaccccgctcttacacattccagccctgaaaaagggcatcaaattaaaccacacctatggtgtatgcatttatttgcatacattcaatcaattgttatctaaggaaatacttacatatgcaagctaaacataaacaacgtaaacgtctgaaatctagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaatctagagtcgacctgcagtaatcgtacagggtagtacaaataaaaaaggcacgtcagatgacgtgccttttttcttgtgagcagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 14 - protein, 115 aa
MQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 15 - DNA (genomic), 15 bp; single-stranded, linear
catgggcagctcgag

SEQ ID NO: 16 - DNA (genomic), 42 bp; single-stranded, linear
gagggttgtgggtgtcgctagtgagtcgactacagcaaaatt

SEQ ID NO: 17 - DNA (genomic), 38 bp; single-stranded, linear
ggatgtgggtgccgctgactctagagtcgacggaattc

SEQ ID NO: 18 - DNA (genomic), 31 bp; single-stranded, linear
aattcaccatgattcctggtaaccgaatgct

SEQ ID NO: 19 - DNA (genomic), 25 bp; single-stranded, linear
cattcggttaccaggaatcatggtg

SEQ ID NO: 20 - DNA (genomic), 30 bp; single-stranded, linear
cgacctgcagccaccatgcatctgactgta

SEQ ID NO: 21 - DNA (genomic), 27 bp; single-stranded, linear
tgcctgcagtttaatattagtggcagc

SEQ ID NO: 22 - DNA (genomic), 15 bp; single-stranded, linear
cgacctgcagccacc

SEQ ID NO: 23 - DNA (genomic), 81 bp; single-stranded, linear
tcgacccaccatgccggggctggggcggagggcgcagtggctgtgctggtggtgggggctgtgctgcagctgctgcgggcc

SEQ ID NO: 24 - DNA (genomic), 73 bp; single-stranded, linear
cgcagcagctgcacagcagcccccaccaccagcacagccactgcgccctccgccccagccccggcatggtggg

SEQ ID NO: 25 - DNA (genomic), 11 bp; single-stranded, linear
tcgactggttt

SEQ ID NO: 26 - DNA (genomic), 9 bp; single-stranded, linear
cgaaaccag

SEQ ID NO: 27 - DNA (genomic), 18 bp; single-stranded, linear
tcgacaggctcgcctgca

SEQ ID NO: 28 - DNA (genomic), 10 bp; single-stranded, linear
ggcgagcctg

SEQ ID NO: 29 - DNA (genomic), 29 bp; single-stranded, linear
caggtcgacccaccatgcacgtgcgctca

SEQ ID NO: 30 - DNA (genomic), 27 bp; single-stranded, linear
tctgtcgacctcggaggagctagtggc